(19) United States
(12) Patent Application Publication
(10) Pub. No.: US 2023/0000378 A1
(43) Pub. Date: Jan. 5, 2023

(54) Method and means to measure oxygen saturation/concentration in animals

(71) Applicant: Sergio Lara Pereira Monteiro, Los Angeles, CA (US)

(72) Inventors: Heather Marisa Richardson, Los Angeles, CA (US); Sergio Lara Pereira Monteiro, Los Angeles, CA (US)

(73) Assignee: Sergio Lara Pereira Monteiro, Los Angeles, CA (US)

(21) Appl. No.: 17/943,114

(22) Filed: Sep. 12, 2022

Related U.S. Application Data
(63) Continuation-in-part of application No. 17/492,655, filed on Oct. 3, 2021, now Pat. No. 11,504,016.
Application No. 17/492,655 is a division of application No. 17/113,035, filed on Dec. 5, 2020, now Pat. No. 11,172,836.
(60) Provisional application No. 62/944,293, filed on Dec. 5, 2019.

(51) Int. Cl. A61B 5/024; A61B 5/00; A61B 5/11

(52) U.S. Cl. CPC A61B 5/02416; A61B 5/683; A61B 5/11

(57) ABSTRACT

We disclose an improvement for oximeters, which makes oximeters more reliable when making measurements on patients of darker skin complexion. The device of our invention discloses a re-entrant cavity, inside which some of the tissues of the patient are forced into, by pressing the device of our invention against the skin of the patient. The probing electromagnetic radiation beams (typically deep red and infra-red radiation) are directed to propagate through said re-entrant cavity, inside which some of the outer tissues of the patient are forced, along a path that is approximately parallel to, and just under, the skin of the patient. This probed volume inside said re-entrant cavity contains more arterial and less venous blood, when compared with measurements made by perpendicular beams, that penetrate deep under the skin, which causes that the measurements made by our device are more accurate than many existing oximeters.

### CROSS-REFERENCE TO RELATED APPLICATIONS

This patent application is a Continuation-in-part of patent application Ser. No. 17/492,655, filing date 2021 Oct. 3, titled “Method and means to measure heart rate with FitBit devices −2” named inventor Sergio Lara Pereira Monteiro, currently under examination, which is a divisional patent application from patent application Ser. No. 17/113,035, filing date 2020 Dec. 5, titled “Method and means to measure heart rate with FitBit devices” named inventor Sergio Lara Pereira Monteiro, now issued U.S. Pat. No. 11,172,836, issue date 2021 Nov. 16. This patent application claims priority over the U.S. provisional patent application No. 62/944,293, dated 2019 Dec. 5, titles “Method and means to illuminate body parts to measure back scattered radiation and to measure heart rate” named inventor Sergio Lara Pereira Monteiro, and to provisional patent application No. 63/329,394, filing date 2022 Apr. 9 , titled “Method and means to measure oxygen saturation in animals”, named inventors Heather Marisa Richardson and Sergio Lara Pereira Monteiro. This patent application is related to U.S. patent application Ser. No. 16/931,407, dated 2020 Jul. 16.

### FEDERALLY SPONSORED RESEARCH

Not applicable

### SEQUENCE LISTING OR PROGRAM

Not applicable

### BACKGROUND OF THE INVENTION

### Field of Invention

This invention relates to FitBit-type devices and to oximeter devices (REF 2), specifically to an improved method and means to measure heart rates and oxygen concentration/saturation of the wearer, and to improve the reliability of the heart rate data acquisition and of the oxygen saturation/concentration, particularly when the wearer is of a dark skin complexion.

### Discussion of Prior Art

For better accuracy and to comply with the USPTO rules, in particular with 35 U.S.C. 112(a) or 35 U.S.C. 112 (pre-AIA), first paragraph, which requires the specification to be written in “full, clear, concise, and exact terms.” also requires that the specification be “clear and full” and the use of “exact terms to enable any person skilled in the art or science to which the invention pertains to make and use the same”, we want to first define a few of the key terms used in the sequel.

Back scattering. This term is used by physicists to mean scattering along a direction that is larger than 90 degrees with the initial direction of propagation, that is, scattering propagating towards the back hemisphere from the point of view of the initial direction of propagation. Cf. forward scattering

Collimator—a device that blocks the path of all light that is propagating outside a certain direction and outside a certain angle of acceptance.

Fitbit—this is a trade name and the name of a company based in San Francisco, but currently used as a noun for any of the “activity trackers, wireless-enabled wearable technology devices that measure data such as the number of steps walked, heart rate, quality of sleep, steps climbed, and other personal metrics involved in fitness” (copied from the FitBit article at Wikipedia, accessed on 2019 Aug. 8). We use the word here in this patent application document as a noun, referring to any device that collects data for physical activity in general, and of the heart rates in particular. In any instance that we intend to refer to the company we will make it explicit, otherwise FitBit here is a noun. As a noun, FitBit means any fitness measurement device, usually wore at the wrist, as a wrist-watch of the fore, which could include a wrist-watch too, which is capable of measure and perhaps also store in computer memory, data related to the physical activity of the wearer. Though most FitBits are designed to be wore at the wrist, it is possible to design and use a FitBit attached to any other part of the wearer's body.

Forward scattering. This term is used by physicists to mean scattering along a direction that is less than 90 degrees with the initial direction of propagation, that is, scattering still propagating towards the forward hemisphere. Cf. Back scattering

Normalization. In mathematics the expression “to normalize” refers to the mathematical procedure of modifying a fraction to an equivalent fraction with a standard (or normal) denominator. It may also mean to describe the quantity based on a standard unit (as meters, or millimeter, or inches, etc.). It often means to multiply all the measurements by the same number, in such a way that the highest number, or value, is some chosen value, most often 1 (one). Probabilities, for example, are normalized to 1, because the highest value for them is 1, by convention.

Oximeters. We use this word here in the ordinary sense that it is used in health care: the devices used to measure oxygen concentration/saturation in the blood stream (REF 2). Most people have been exposed to the oximeters, particularly in our post-COVID word, most medical facilities currently using oximeters that resemble a cloth-pin, which the nurse, or other medical practitioner, clips in our fingers upon entrance. I assume that occasionally, after reading the result, the nurse jumps back and run away, which is followed by a half-a-dozen big men, completely covered in plastic, coming in to remove the person. This never happened to me, I just assume that it does occur. Oximeters are not necessarily clipped on the finger of the patient; it could be any other location.

Transmitted light. In this patent application we will be using the words “transmitted light” in a meaning that is different than the meaning it is normally used. By “transmitted light” we mean here the light that is transmitted without suffering any scattering event.

We discuss here the heart beating measurement and oxygen concentration/saturation capability of the FitBits in general, also more specialized devices, constructed exclusively to measure the oxygen concentration/saturation. For example, oxygen concentration/saturation is an important information regarding COVID, because the CoronaVirus decreases the lung capability and ability to oxygenate the blood. Consequently, since the pandemic started, it became common practice to use devices called oximeters, which measure the amount of blood oxygenation, usually in percentage of some value, which, in its most common form look as a cloth pin, which the nurse clips on the patient's finger, usually the pointing finger. These oximeters work Our invention is an improvement on the heart rate measurement and on the oxygen concentration/saturation capability of the FitBits and/or more specialized devices, as the cloth-pin devices so common after COVID came in. In the sequel we will be referring to fitbit devices, it being understood that the device in question could be more narrowly specialized to measure only and exclusively oxygen concentration/saturation.

Existing FitBit-type devices are well known to often fail to make any acceptable measurement the heart rate of darker skinned people—brown and black. This is well documented in the literature, it being easy to find plenty of documented shortcomings for the measurement of the heart rate and/or the blood oxygen saturation/concentration on darker-skined people, if one just googles any of the terms related to it, e.g. “FitBit heart rate darker skin”, or “oximeter darker skin”. Such web search easily find a large number of complaints from darker skinned people, who consistently report that their FitBit-type devices make terribly wrong measurements of their heart rate, wrong when compared with the old-fashioned method of measuring the beats at the pulse with a finger pressed on the wrist and counting the beats for a known length of time. In fact, it is reported that the FitBit heart rates of darker skinned people is sometimes as low as 20 or 30, which is an obviously wrong number if the individual in question is not fainting. This failure of the FitBit-type devices is so well known that it has been measured and published in fitness magazines and published by established university research teams in science journals as well. Equivalent findings have been reported with respect to the results obtained by the oximeters, when measuring the oxygen saturation on darker skinned persons.

Before we proceed, let us add a few words of how the heart beat is measured by the FitBits. It uses a process known in physics as light spectroscopy, which is the process of measuring the light energy that is either absorbed or scattered by a particular molecule. Measuring either scattering or absorption of some molecule present in blood, for example, while not present in other human tissues, gives information about either the amount or some other information on blood in the volume illuminated by the light—because blood and only blood can cause the change, in such a situation. In this case, where the objective is the determination of the heart beating of the wearer of the FitBit, another feature is also used, that with each heart contraction (systole), the amount of blood in the arteries increases, decreasing after, as the blood continues to fill the veins and be recollected by the heart, (diastole). In short, the FitBit measures the amount of blood at a particular place on the body of the wearer, which, in turn, increases and decreases as the heart pumps the blood. If this can be measured, it follows that each cycle corresponds to a single heart beat. To advance the conclusion later, the problem with the existing FitBit is that they are poor at measuring the blood amount when the FitBit wearer has darker skin, because the melanin absorbs the light (REF 1), so there is no light inside the body to measure anything! The same, mutatis mutandis, is valid for the oximeters. The former was the subject of issued and allowed patents, the later is the subject of this patent application here.

It is interesting to notice here that spectroscopy is used by our brains to make sense of the world as well, it being how we detect that a leaf is a leaf, that there is a wall ahead of us, so we better not proceed ahead, or that a person is white, brown or black, so white Americans can avoid marrying with the latter two groups. This is so because part of the information used by our brain is the color of the light scattered towards our eyes: green for light reflected by the leaves, yellow for the light reflected by the hair of a blond person, etc. The last example is a personal observation of one of the inventors (SLPM), involving personal perception of the reality.

The FitBits and the oximeters use the same method. In this case, the device is fitted with a light of a particular color, usually green for the FitBit case, or a combination of deep red and 940 nm (infra red) for the oximeter, which is turned on from the base of the device, into the wrist or other body part of the wearer, and a green light detector, or red/near infrared light that measures the light reflected back or transmitted onto the FitBit. The only difference between the FitBits and our eyes, being that our eyes are incapable of emit light, so they use the light emitted by the sun, or else the artificial light reflected by the wall of a room we happen to be in, while the FitBit produces it own light, but this is a trivial difference that does not change the nature of the process. It is because the FitBit measures the back-scattered light that the method to measure the heart beating rate is called spectroscopy. Some devices, particularly a large number of oximeters, use transmitted light, instead of back-scattered light, but this detail does not change more than the details of the process. Depending on the molecule used by a particular FitBit, there will be a certain fraction of the light that is back-scattered onto the FitBit, which is, in turn, a function of how many of these molecules are in the light path. The measurement is then based on the fact that the amount of blood in the blood vessels change during the heart beating process: more blood at the pressure peak (systole), when the blood is pushed hard into the arteries, less blood at the low part of the cycle (diastole), when the blood flows away from the vessels into the heart. So, when there is more blood, there is more of the particular molecule that is measured, and consequently more light back-scattered, and less light is transmitted. In such a situation, the microcontroller in the FitBit is looking for how many times per minute the light intensity goes up and down, then up again and down again, etc., each cycle corresponding to a systole-diastole cycle, which, in turn, corresponds to a heart beat, so measuring the number of cycles, the FitBit is measuring the heart beats. This works as long as there is enough photons (or particles of light) to make each measurement. If, because of melanin absorption for example, the number of photons is too small, then the detection of the maximum-minimum becomes uncertain, and with this so does the number of heart beatings (REF 1).

Now for the case of our interest here, it is generally accepted that the error of the heart rate reading for darker skinned people, when compared with the same measurement, by the same device, on lighter skinned persons, is due to the use of green light, which is more absorbed by the extra melanin on the skin of the darker skinned individuals, when compared with the less melanin on the skin of the lighter skinned individuals. This green light is used to acquire the data but it hardly penetrates beyond the skin of a dark-skinned person, which is composed of dead cells, being therefore unable to reach the blood underneath the skin, to allow for the measurement of the required data, in this case, the green light intensity that is back-scattered (that is reflected backwards, in non-technical language).

To understand the failure, one needs to look at the measurement process used by the devices. In most existing FitBits, with small variation for the oximeters, the measurement of the heart beat is made using a green light beam, which is sent into the body of a person, usually the wrist, then measuring the intensity of the light back-scattered (reflected back, in ordinary language). Leaving aside the details, the FitBit process works similarly to the way our brain uses to detect the presence of blood on a wound or skin cut, or a subset of the information used by our brain: a measurement of the back-scattered light intensity of the color red. It stands to reason that for this to work it is necessary that the probing device (the green light) must be able to reach the blood to be measured. It is known that blood absorbs more red and infrared (IR) light than flesh does, and also scatters more red and infrared (IR) light than flesh does, including back-scattering. This differential optical property between blood and flesh can be used to measure the variation of blood that accompanies each and every heart beat. Here we want to highlight the use of a technical term that is easy to understand by a physicist but may be misunderstood by someone with less acquaintance with scientific use of words. When a physicist writes, as I did, “This differential optical property . . . ” we mean “the existence of this difference on the optical property . . . ”, and also by “optical property” we physicists include at least infrared and ultraviolet with the optical properties, if not beyond these too. It is also worth to point out here, that though the existing FitBits are better at measuring the heart rate of fair skinned people, when compared with darker skinned people, the heart rate measurement even for the fairer skinned people leaves a lot to be desired; in other words, the FitBits do a better job to measure the heart rate for fair skinned people but they still leave a lot to be desired even for the fair skinned people.

This failure of the FitBit devices to correctly measure the heart bit rate on darker skinned people, is said to be a consequence of the devices using green light to measure the amount of blood at the wrist, which blood supply increases and decreases just below the skin at the wrist, with each heart beat, and with each in-between beat, respectively. The reason that the FitBits fail for darker skinned people is because darker skinned people have more melanin on their skins, which absorbs enough green light that there are not enough green photons to make a measurement, so the FitBits fail for darker skinned people. The oximeters, on the other hand, use two “colors”, deep red and infrared.

On the other hand, it is well known that infrared radiation is capable of penetrating deeper into tissues, including melanin, a point that has been pointed out to the FitBits manufacturers. These manufacturers, in turn, reply that they cannot use red/infrared, instead of green, because of the higher price of the red and/or infrared sources. The inventor knows well that this larger price of the green LED and/or lasers is not true, and so do most of people familiar with the subject of LEDs and laser diodes. This then brings to the fore the question: what is then the reason to use green light? Why do the manufacturer of FitBits lie (this is one of the inventors constitutionally protected personal opinion, not a statement of fact)? The true reason is that it is better to measure changes of blood irrigation (blood supply) just under the skin, as opposed to measure changes of blood supply deeper below the skin. Of course that this, in turn, poses another question: why are the FitBit manufacturers lying (again, this is one of the inventors constitutionally protected personal opinion, not a statement of fact)? The inventors believe that the FitBit manufacturers are holding as a trade secret that they use the green light to probe just below and not too deep under the skin, for the reason described in the sequel. Instead of making it public and patenting it, the crucial information that what matters most is the change of superficial blood supply, as opposed to deeper blood supply. Though this is the conclusion of the inventors, this particular observation should not be held against the patent application, because the method and means disclosed in this patent application do not depend on the going-ons inside the manufacturers of FitBits, or on the theoretical explanations of the device, but only that the method and means work, as measured by the inventors.

### OBJECTS AND ADVANTAGES

It is one of the objects and advantages of this invention to provide an improved measurement of the heart rate as measured by FitBit-type devices when the wearer of the FitBit-type device is of a darker skin color.

It is another object and advantage of this invention to provide an improved measurement of the heart rate as measured by FitBit-type devices even when the wearer of the FitBit-type device is of a lighter skin color.

It is another object and advantage of this invention to provide an improved measurement of the oxygen concentration/saturation as measured by oximeter devices, particularly when the wearer of the oximeter device is of a darker skin color.

It is another object and advantage of this invention to provide an improved measurement of the oxygen concentration/saturation made by oximeter devices even when the wearer of the oximeter device is of a lighter skin color.

It is another object and advantage of this invention to bring some dough to the inventors to pay-off our mortgages and also pay-off Diane's farm.

If one or more of the cited objectives is not achieved in a particular case, any one of the remaining objectives should be considered enough for the patent disclosure to stand, as these objectives are independent of each other.

### SUMMARY OF THE INVENTION

Our invention discloses the use of infrared radiation, which can penetrate the melanin better than green radiation does, and ALSO directs the infrared radiation to a path parallel to and just under the skin, which prevents deep penetration of the infra-red, because there is more variation of blood supply just below the skin than deep below the skin. Our beautiful invention that will revolutionize the capability of the FitBits and the oximeters, to correctly measure the heart rate and the oxygen saturation of the wearer, discloses the use of a light beam propagating just below the skin surface, at a direction substantially parallel to the skin surface, to measure the blood volume. Forcing the radiation probing beam (red and infrared in the main embodiment) to be just under the skin, the device of this invention forces that the measurement occurs at the place where there is larger volume of blood variation, for the case of the FitBit, and forces that the measurement occurs where there is more blood that has just been ejected by the heart, or fresher blood, so to say, therefore maximizing the resultant measurement, for the oximeter. This is so because if the measuring photons (particle of light) penetrates deep into the body, then the photon path is along volumes with more venous blood, which is blood which is less subjected to the pressure oscillations due to the heart beatings, in the case of the FitBit, or else its path that contains less oxygenated blood, in the case of the oximeter, both being undesirable situations. The light used should preferentially be either deep red or infrared, for the FitBit, and be two beams, of different wavelengths, usually deep red and 950 nm, for the oximeter, and the beam should preferentially be a collimated beam, as a laser beam, but these are not necessary for the invention to work, but only to work better. For the oximeter, for example, it is possible to use different pairs of lights, other than red and 950 nm, or even more than two wavelengths, there existing devices that use 5 different wavelengths.

This invention also discloses a collimator that rejects “light” beams that propagate toward the detector along directions not intended to be measured, as, for example, to eliminate scattered photons before they reach and are detected by the “light” detector that is intended to measure the “transmitted” photons, which are the photons that have propagated along the original directions without suffering any scattering event—as per MY DEFINITION of the term, which is my definition used here for convenience and not any usual or accepted terminology. Indeed, without such a collimator many photons would reach the detector that is measuring the “transmitted” radiation that are not “transmitted” photons, but rather are scattered photons propagating toward the detector positioned to measure the “transmitted” radiation but that do propagate along the initial direction of propagation after multiple scattering events, then reach the detector from another direction other than the initial propagating direction, or, in other words, that are not directly along the initial direction of propagation.

### BRIEF DESCRIPTION OF THE DRAWINGS

FIG. 1A. is a perspective view of our invention.

FIG. 1B. is a cross section on the perspective view seen at FIG. 1A at the vertical plane A-A′.

FIG. 2. Fitbit-type supporting structure shaped to encase itself around the wearer's wrist with the objective of emitting a radiation beam LB that crosses the window WIN then penetrates the body just under the skin SK. Beam LB may suffer scattering events at scattering center SC (SP), being partly scattered forward, partly scattered at almost 90 degrees, to be detected at radiation detectors LD1 and LD2.

FIG. 3. Fitbit-type supporting structure shaped to encase itself around the wearer's wrist with the objective of emitting a light beam LB that crosses the window WIN then penetrates the body at normal (90 degrees) incidence. Beam LB may suffer scattering events at scattering center SC (SP), being partly scattered forward, partly scattered at almost 90 degrees, to be detected at light detectors LD.

FIG. 4. Fitbit-type supporting structure shaped to encase itself around the wearer's wrist with the objective of emitting a light beam LB that crosses the window WIN then penetrates the body at normal (90 degrees) incidence. Beam LB may suffer scattering events at scattering center SC (SP), being partly scattered forward, partly scattered at almost 90 degrees, to be detected at light detectors LD.

FIG. 5. Fitbit-type supporting structure shaped to encase itself around the wearer's wrist with the objective of emitting a light beam LB that crosses the window WIN then penetrates the body at normal (90 degrees) incidence. Beam LB may suffer scattering events at scattering center SC, being partly scattered forward, partly scattered at almost 90 degrees, to be detected at light detectors LD.

FIG. 6. Several elements of our FitBit-type device of our invention.

FIG. 7. Very, very old art, really old art! A wrist-watch type device adapted to show the time. Time keepers became important with the open ocean navigation and invasion of the Americas, because latitude, or north-south position, was always possible to determine easily by observing the height of certain stars above the horizon (say, the Northern Star), but the longitude could not be determined without the knowledge of the time at some reference place (say Greenwich or Paris), so a true clock or watch was crucial for navigating the open ocean, else the ship could run onto the shore at night and kill everybody on board.

FIG. 8A. RFitBit=Re-entrance on FitBit, SFitBit=Surface of FitBit. View of the device of my invention away from skin surface.

FIG. 8B. RFitBit=Re-entrance on FitBit, SFitBit=Surface of FitBit. View of the device of my invention touching skin surface

FIG. 8C. RFitBit=Re-entrance on FitBit, SFitBit=Surface of FitBit. View of the device of my invention pressed against skin surface; skin adapts to contour of device of my invention, filling-in the re-entrant “hole” of the device of my invention.

FIG. 9A Fitbit type supporting structure shaped to encase itself in the wearer's body with the objective of emitting a light beam LB that crosses the window WIN then penetrates the body at normal (90 degrees) incidence.

FIG. 9B. Laser emitting light beams redirected by total internal reflection at lower (inner) side of sf and normal (perpendicular) light detectors ahead of beam.

FIG. 10A. Laser emitting light beams at grazing angles with skin.

FIG. 10B. Laser emitting light beams at grazing angles with skin with normal (perpendicular) light detectors ahead of beam.

FIG. 11A. Laser emitting light beams redirected by total internal reflection at lower (inner) side of SF.

FIG. 11B. Laser emitting light beams redirected by total internal reflection at lower (inner) side of SF and normal (perpendicular) light detectors ahead of beam.

FIG. 12A. Fitbit-type supporting structure shaped to encase itself in the wearer's body with the objective of emitting a light beam LB that crosses the window WIN then penetrates the body at normal (90 degrees) incidence.

FIG. 12B. Laser emitting light beams redirected by total internal reflection at lower (inner) side of SF and normal (perpendicular) light detectors ahead of beam.

FIG. 13A. 90 degrees scattering from a light beam LB propagating just below the skin SK.

FIG. 13B. 180 degrees scattering.

FIG. 14. Detail showing light emitter LE, light beam LB, surface SF and window WIN. Window WIN is preferably perpendicular to the light beam LB, as shown here.

FIG. 15. Light detector of our invention with the detecting element at the end of a cylinder that acts as a collimator COL, preventing “light” from reaching the light detecting element LD unless it is propagating along a certain preferred direction (within a defined angular aperture).

FIG. 16. Side view of one type of supporting structure SS of our invention with the protruding blocks PB1 and PB2 of depth d, Light emitter LE and light detector LD, a first propagation path FPP of the light beam and second propagation path of the light beam, the re-entrant cavity RC

FIG. 17. Side view of a second type of supporting structure SS of our invention with Light emitter LE and light detector LD, a first propagation path FPP of the light beam, a second propagation path SPP of the light beam, the re-entrant cavity RC of depth d.

FIG. 18. light emitter LE at left amitting photons along the first propagation path FPP to the right.

FIG. 19. light emitter LE at left amitting photons along the first propagation path FPP to the right.

FIG. 20. See cloth-pin supporting structure CP, re-entrant cavity on one or both arms of device, one or more light emitters LE, one or more light detectors LD, positioned to detect transmitted light and/or scattered light. Spring mechanism to keep CP closed on finger or other part of patient not shown, SA=separator axle/d=depth of re-entrant cavity/SCD=spring.

FIG. 21. See cloth-pin supporting structure CP, re-entrant cavity on one or both arms of device, one or more light emitters LE, one or more light detectors LD, positioned to detect transmitted light and/or scattered light. Spring mechanism to keep CP closed on finger or other part of patient not shown.

### DRAWINGS—LIST OF REFERENCE NUMERALS

### Brief Description of Labels

BOX=SS=holding and containing device, or box or container, or supporting structure SS, which contains the elements of our invention.
infrared (IR)=infrared radiation (IRR). We are using the term as it is understood in physics, all radiation characterized by wavelengths longer than deep red, beyond the visible spectrum.

This is how Wikipedia defines it, as assessed on 2020 Nov. 29:
Infrared—Wikipedia
en.wikipedia.org>wiki>Infrared
Infrared (IR), sometimes called infrared light, is electromagnetic radiation (EMR) with wavelengths longer than those of visible light.
FPP=First propagation path or direction of propagation, or direction.
LB=Light (radiation) Beam
LD=Light (radiation) detector
LE=Light (radiation) Emitter
PB=Protruding block
PB1=Protruding block1
PB2=Protruding block2
radiation=we are using this term as it is used in physics, a short for electromagnetic radiation

(EMR), which has nothing to do with cancer causing radiation, as it is assumed by most people. Don't be afraid of radiation here guys, this is physics radiation.
RC=Re-entrant cavity
RFitBit=Re-entrance on FitBit,
SC=scattering center
SC1=scattering center 1
SF=surface
SFitBit=Surface of FitBit
SK=skin
SP=SC=Scattering point or scattering center
SS=Supporting structure=holding and containing device
ST=strap
STH=strap holder
WIN=window, optical window

### DETAILED DESCRIPTION

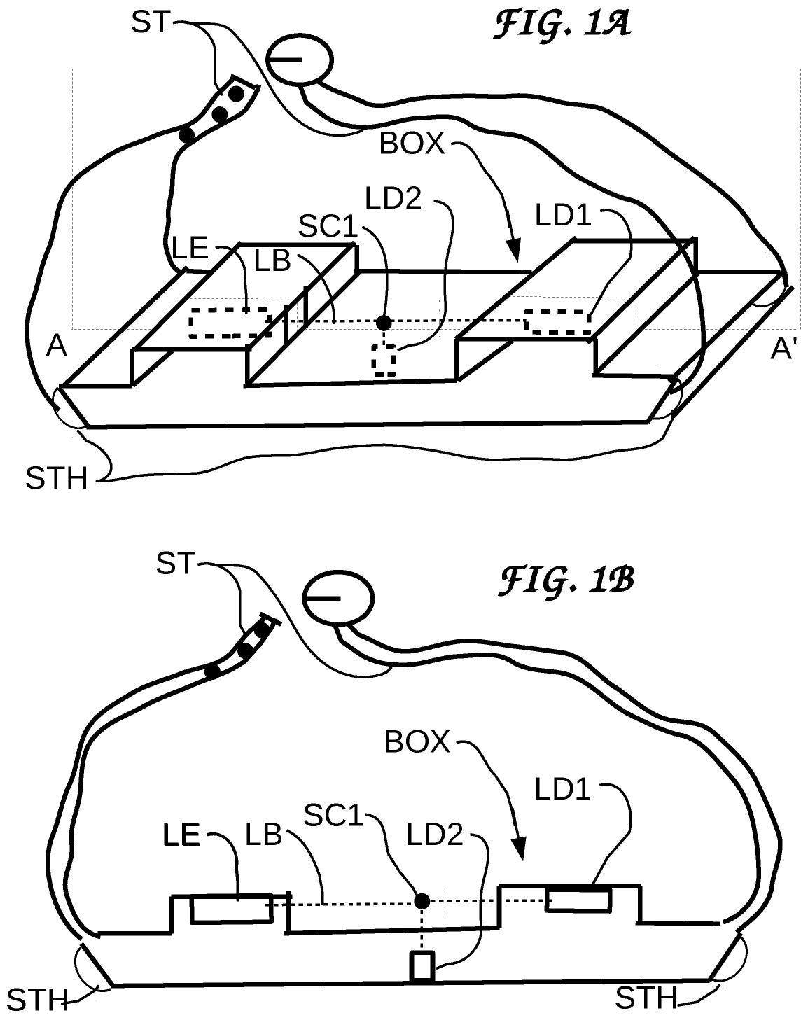

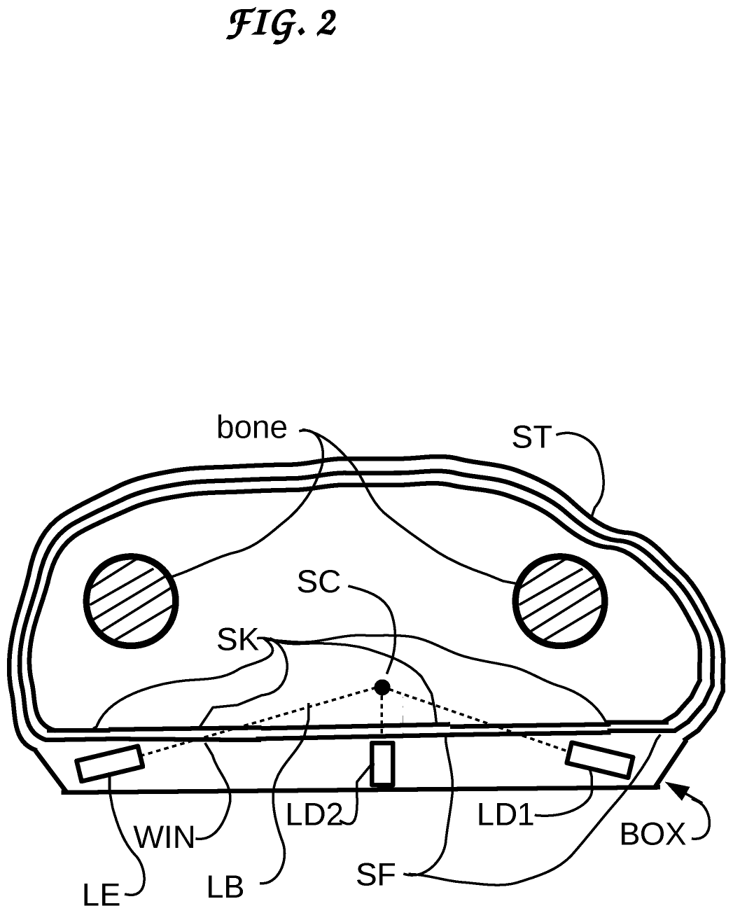

Referring to figures FIG. 1A and FIG. 1B, the main embodiment of our wonderful invention for the FitBit case, it is a holding box or container (box), or holding and containing device, or supporting structure SS, with strap holders (STH) capable of keeping attached in place straps (ST) adapted to keeping the container (box) in place and tight held against the wrist of the wearer, similar to the straps that keep wrist-watches in place. The straps are not necessary for the invention, but only one of the possible variations of the invention, because the holding and containing device may be held by hand against the skin SK, for example, or any other method, as the cuff for a blood pressure measurement device, or a ring inserted on one of the fingers of the animal, and etc. For the FitBit case, the main embodiment has two protruding blocks PB, or volumes or wedges, or protruding elements, which are capable of holding in place a radiation emitter LE (for Light Emitter) in a first protruding block PB1, which emits a radiation beam LB (for Light Beam), and a radiation detector LD1 (for Light Detector 1) in a second protruding block PB2, capable of measuring the amount of energy, or the intensity, or the number of photons, of the radiation beam LB that reaches the radiation detector LD1. We call the two protruding blocks as a set as PB. We call the volume between the protruding blocks as a re-entrant cavity RC. Some radiation that is emitted by the radiation emitter LE may be either (1) absorbed by or (2) scattered by the flesh or any cell, particularly the red blood cells circulating in the blood of the animal wearing the device. Radiation detector LD1 is capable of measuring the intensity of the radiation LB emitted by the radiation emitter LE that propagates through the flesh of the animal, including humans, that is pressed down into the space between the protruding blocks PB that hold the radiation emitter LE and the first radiation detector LD1. The main embodiment is also capable of holding in place an optional second radiation detector LD2 (for Light Detector 2), which is located at such a place that it is capable of detecting radiation scattered out of the main radiation beam LB by the scattering center SC1 (for Scattering Center 1) towards the position of radiation detector LD2. Other detectors at different positions and angles are also possible to be included. Other shapes of the re-entrant cavity are equally possible, as with the smaller sides at some angle different than 90 degrees with the longer dimension, or with curved shape re-entrant cavity, there included spherical, ellipsoid, and no-named curve shapes. It is also possible to have the surface of the supporting structure SS as a single flat surface (no re-entrant cavities, no protruding blocks), while positioning the radiation emitter LE at a shallow angle with the box's surface (that is, with the skin of the wearer) and the radiation detector LD at a shallow angle as well, as seen at figure FIG. 2. The supporting structure SS is also known and referred to as a holding and containing device. Said supporting structure SS, or holding and containing device, is a structure that keeps in place and with the correct alignment, some or all of the elements of our invention, as the light emitter LE, the light detector LD, a possible collimator COL, including possibly a strapping or wrapping device and other ancillary parts that are necessary for the functioning of the device of our invention.

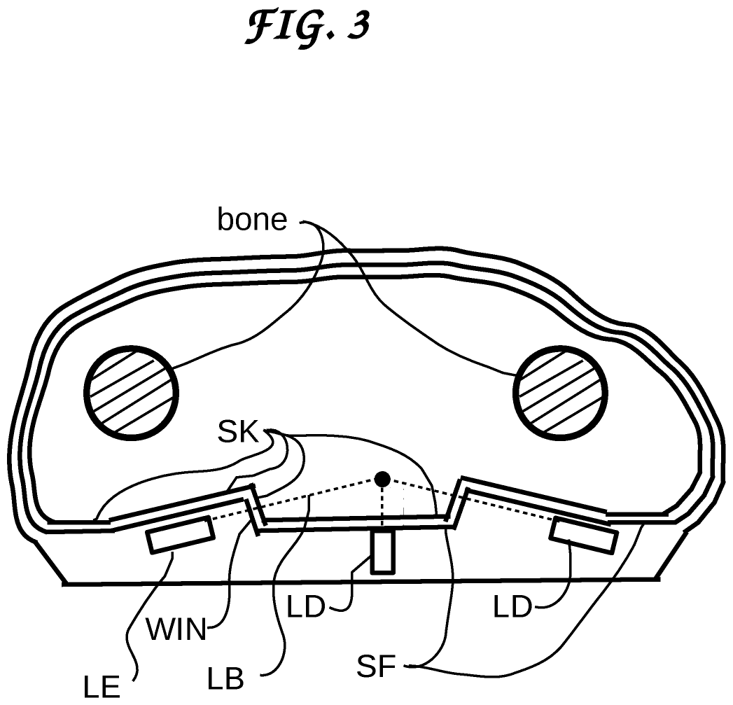

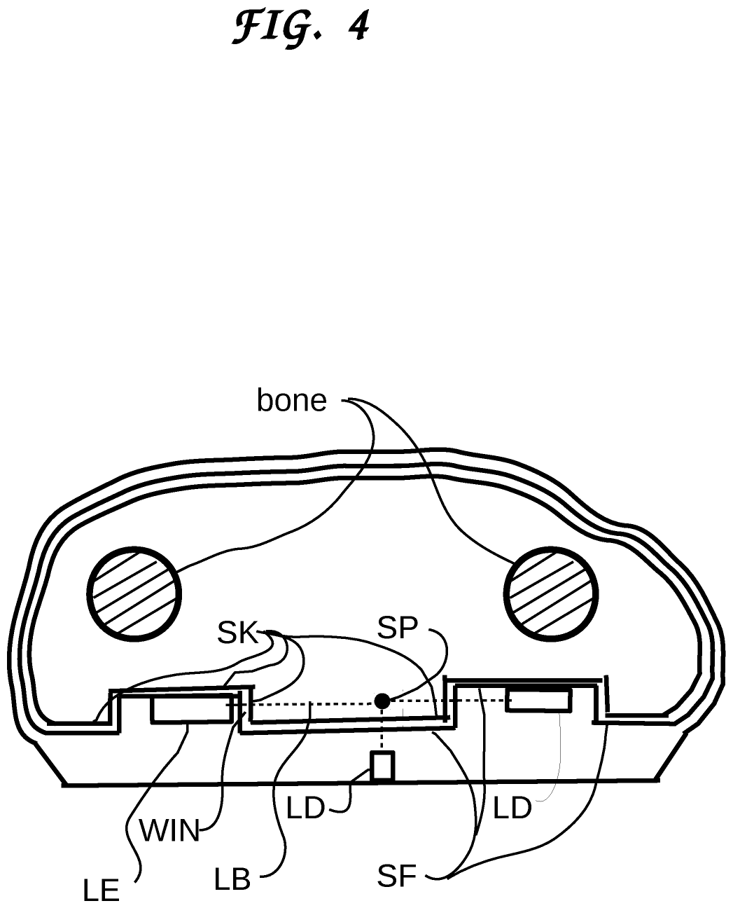

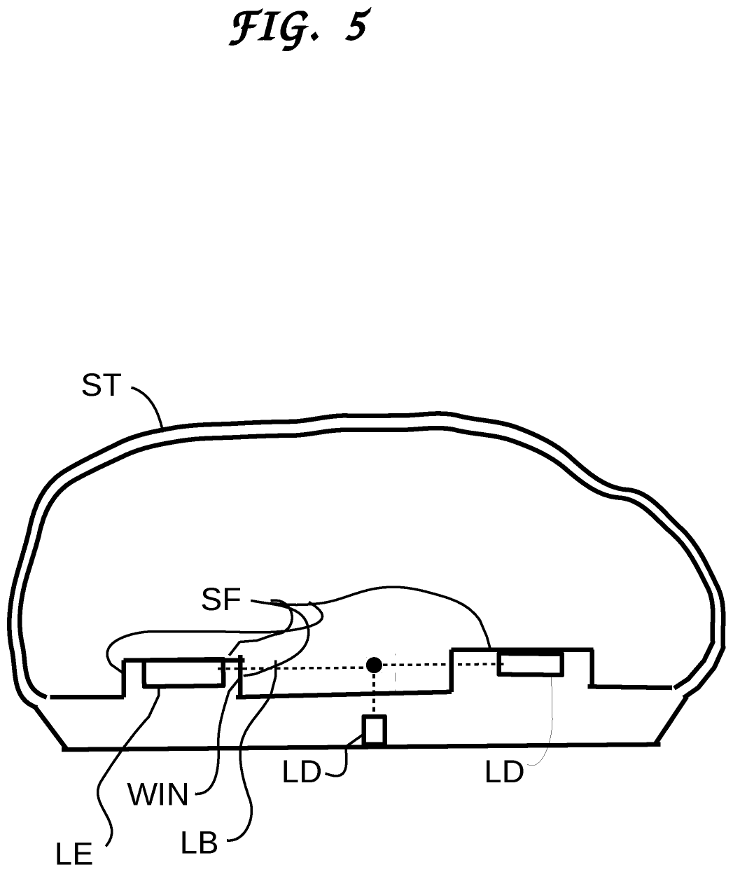

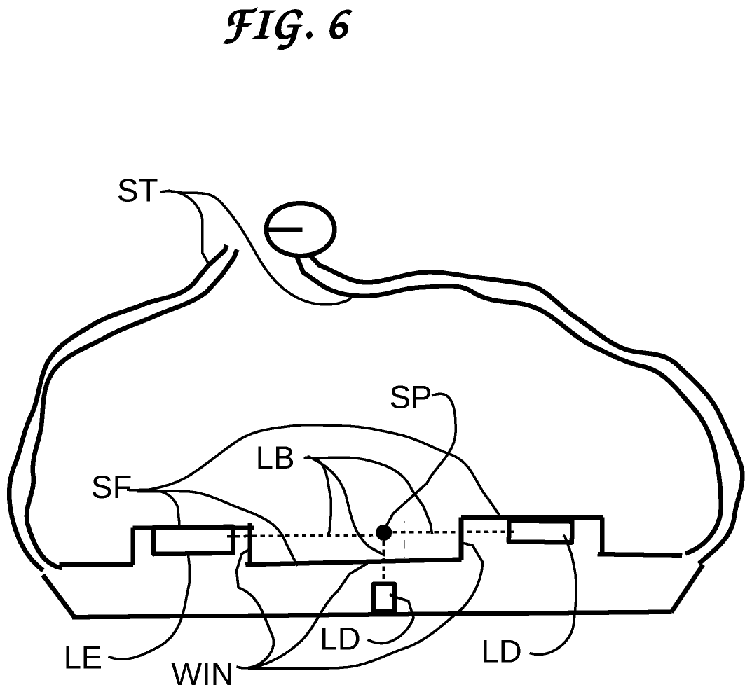

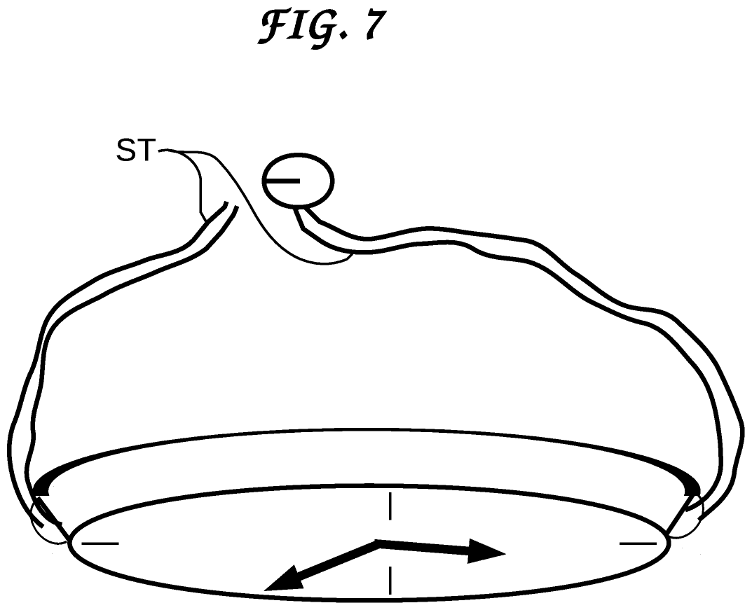

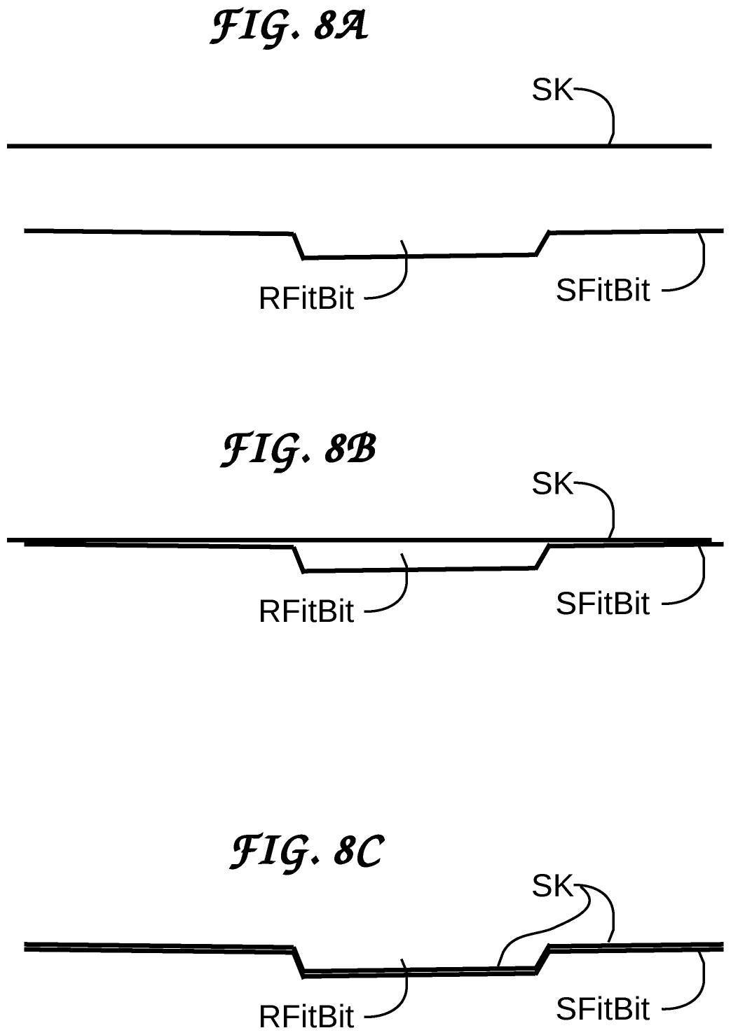

Figures FIG. 3, FIG. 4, FIG. 5, FIG. 6, FIG. 1A, FIG. 1B show several views of the main embodiment of out wonderful invention, which main embodiment is for the FitBit. These figures are all assuming a particular possible embodiment of our invention, which is for use strapped on the wrist of a humans, similarly to a wrist watch, similarly to most fit-bits, as depicted at figure FIG. 7. It is worth to refer to FIG. 7, a wrist-watch, for the reader to keep in mind the structure and size and possible meaning of each element, yet the invention is not restricted to be worn on the wrist, it being possible to position the device of our invention in other parts of the body as well, as around the upper arm, around the elbow, around the upper or lower leg, etc. Referring to FIG. 1A, one can see a perspective view of the main embodiment, for the FitBit, of our invention, NOT attached to any animal or humans. Looking at figure FIG. 1A one can see that our wonderful invention sports two protruding blocks PB, one which contains a light emitter LE, the other that contains a light detector LD1, also referred to as radiation emitter and radiation detector, because the “light” may be other electromagnetic radiations beyond the visible light. The reason for the protruding blocks existance can be understood looking at figure FIG. 4, which also depicts our amazing invention, this time strapped on the wrist of a human. In this figure FIG. 4, which depicts a cross-section of an arm of a human, at the distal end of the arm, which is the normal position for a wrist watch or a fit-bit, one can see the two bones, radius and ulna, and our amazing invention strapped on the wrist, pressed in place, which then causes that the flesh of the human penetrates the re-entrant cavity between the light emitter LE and the light detector LD1. The reader is invited to follow the line SK of the skin of the wearer, and the line SF of the surface of the device of our amazing invention; the skin line SK follows closely the surface line SF because the supporting device of our invention is pressed against the wrist of the wearer. An idealized situation of this is shown at figures FIG. 8A, FIG. 8B and FIG. 8C. In these figures FIG. 8A, FIG. 8B and FIG. 8C the reader can see the skin of the animal away from the surface of the device of our invention at figure FIG. 8A, then the device of our invention just touching the skin of the animal, but not being pressed against the skin, at figure FIG. 8B, then, finally, the device of our invention pressed against the skin of the animal at figure FIG. 8C, which causes that the flesh of the FitBit user penetrates the re-entrant cavity RfitBit, the re-entrant cavity being the volume between the two protruding blocks PB1 and PB2 that hold and support the “light” emitter LE and the “light” detector LD. The reader is reminded here that the re-entrant cavity RfitBit may be a “hole” or cavity, on the inner surface of the supporting structure SS touching the skin of the animal, instead of a volume between two protruding blocks PB protruding out of the inner surface of the supporting structure SS, Naturally that for such a penetration to occur, the depth of the re-entrant cavity needs to be small or shallow, say, 1 mm, or 2 mm, other values, larger and smaller being still compatible with our beautiful invention, without changing the character of it. This last case shows the flesh of the animal penetrating the cavity RFitBit. This penetration is necessary to cause that the radiation beam LB propagates through the flesh of the person who wants his heart beat measured. Naturally that a similar situation occurs for the application to the oximeters, which is a variation described in more detail further down on this specification.

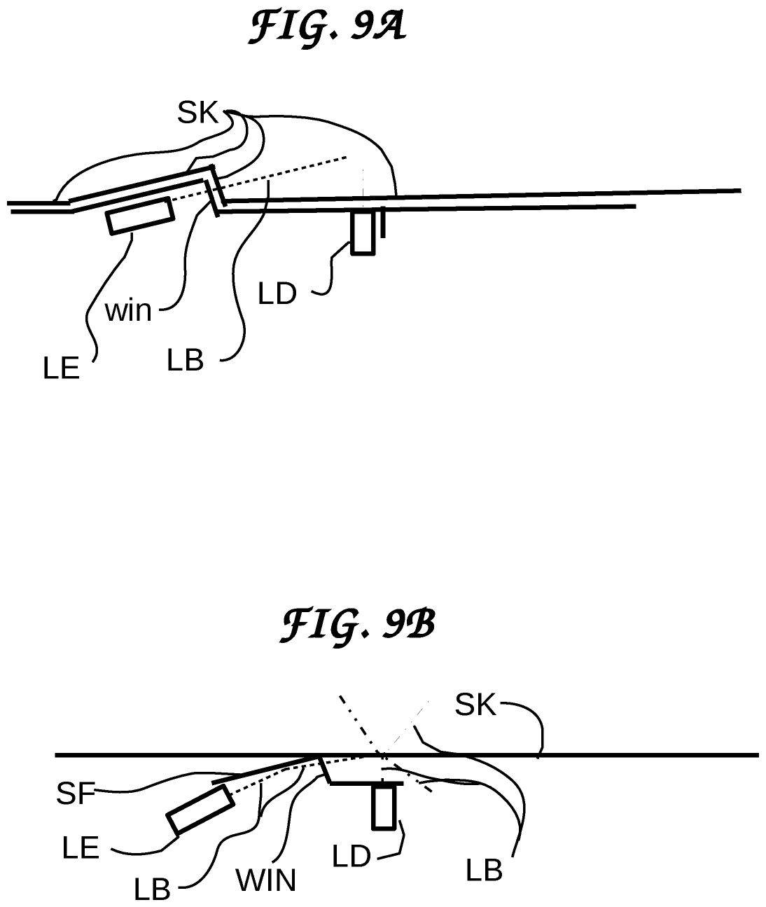

If it is desired to measure the decrease of radiation along the incident beam direction, then the exit window near the radiation emitter LE and the window near the radiation detector LD1 should preferably, though not necessarily, be made perpendicular to the propagation of the radiation. This is not necessary, but it is one of the options protected in this patent. This is the preferred embodiment, as shown in figures FIG. 4, FIG. 1A and FIG. 1B, among others. But it is possible to have the window at such an angle that the radiation beam LB reaches the window at an angle other than 90 degrees, as shown at figures FIG. 9A and FIG. 9B, among others.

It is possible to make the measurement of blood volume using either the scattered radiation or using the radiation that suffered neither scattering not absorption events, which I call transmitted radiation—a non-standard use of the word, my personal use here, not used by anyone anywhere else in my city, in the country where I live, in this planet, in our galaxy, or in the universe at large. Both of these are shown at figure FIG. 4, FIG. 6, FIG. 1A and FIG. 1B. Here it is a good time for a warning to the reader: you should keep in mind that typically both the scattering cross section and the absorption cross section go up-and-down together (remember that cross section is the physics speak for probability of). This means that where there is larger scattering (more radiation impinging on the off-incident direction radiation detector) there is also more absorption, which means that the transmitted radiation is smaller, since the initial radiation beam is decreased by both scattering and by absorption as well. This is most important, it meaning that the scattered radiation measurement is positively correlated with the “opacity” of the scattering centers, while the “transmitted” radiation measurement is NEGATIVELY correlated with the “opacity” of the scattering center!, and, saying it in different words: when the scattered measurement goes up in most cases the “transmitted” measurement goes down!, or, in still different words: the scattering measurement is a positive image of the points, while the “transmitted” measurement is a negative image of the points. The points together form the images, one which I call “scattered image”, and other that I call “transmitted image”, which are the negative of each other. They cannot be summed up to obtain a complete image! They can be joined into an image that uses all the information, but a mathematical manipulation needs to be performed on one or the other, before merging them!

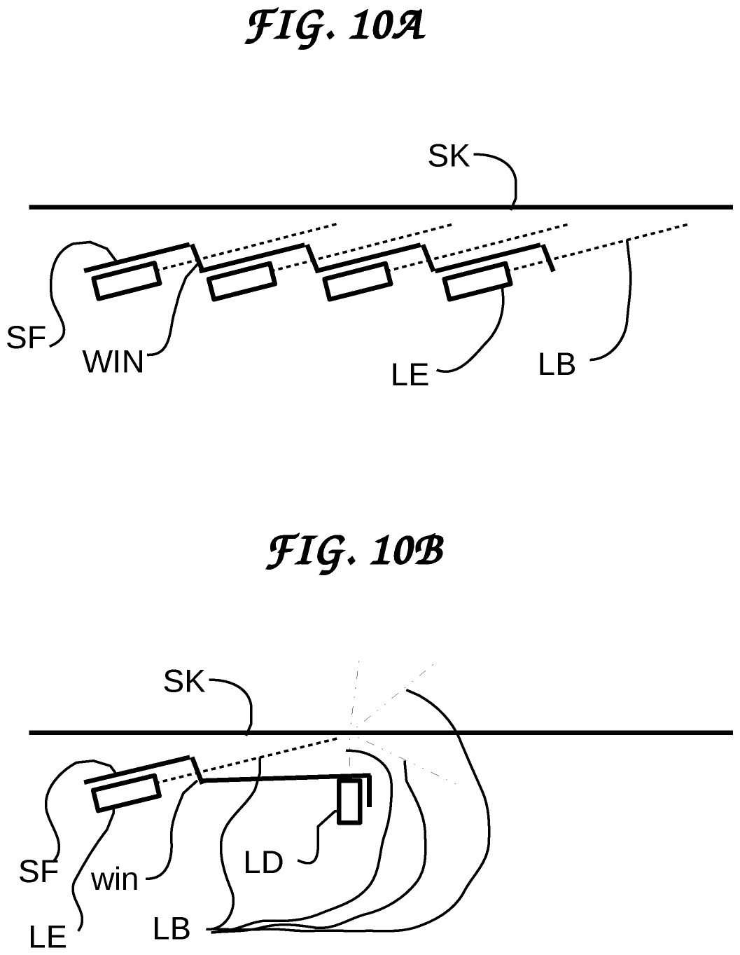

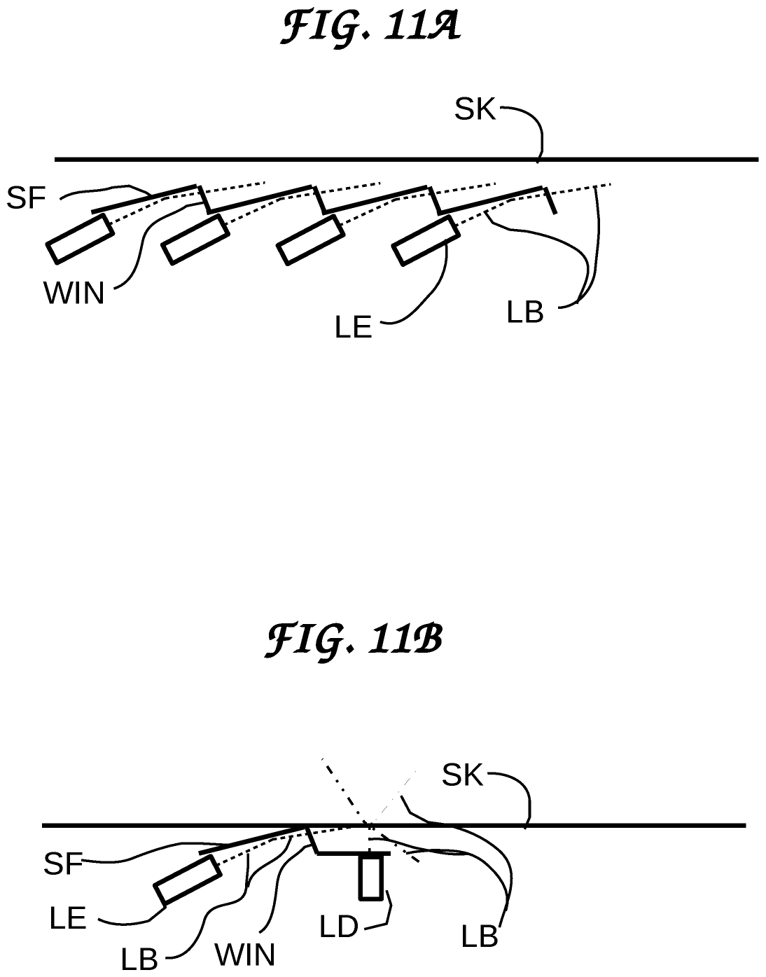

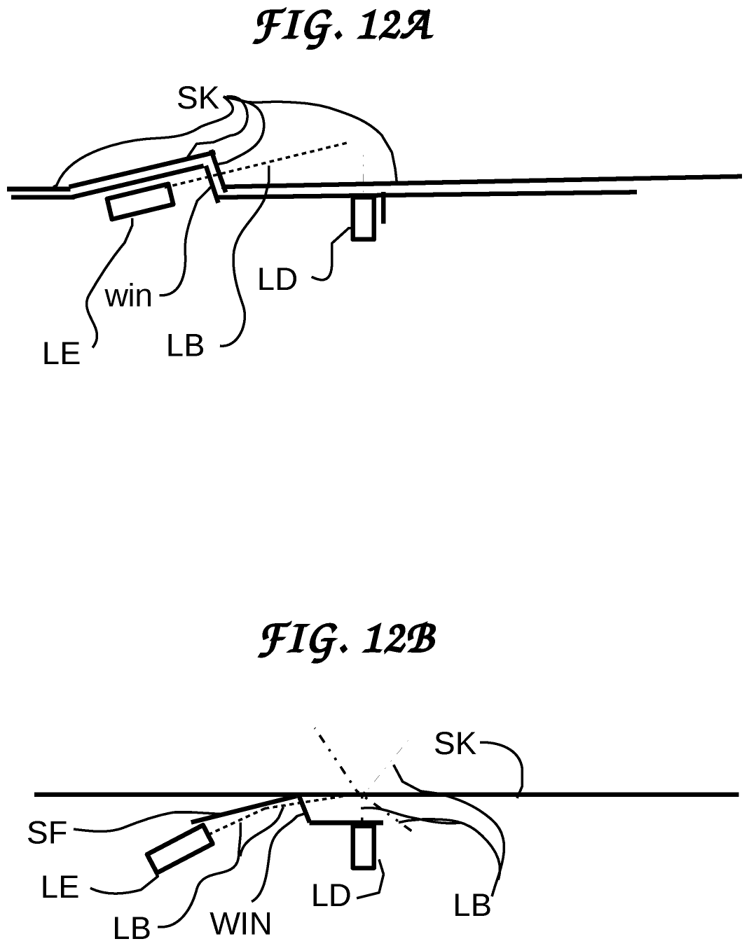

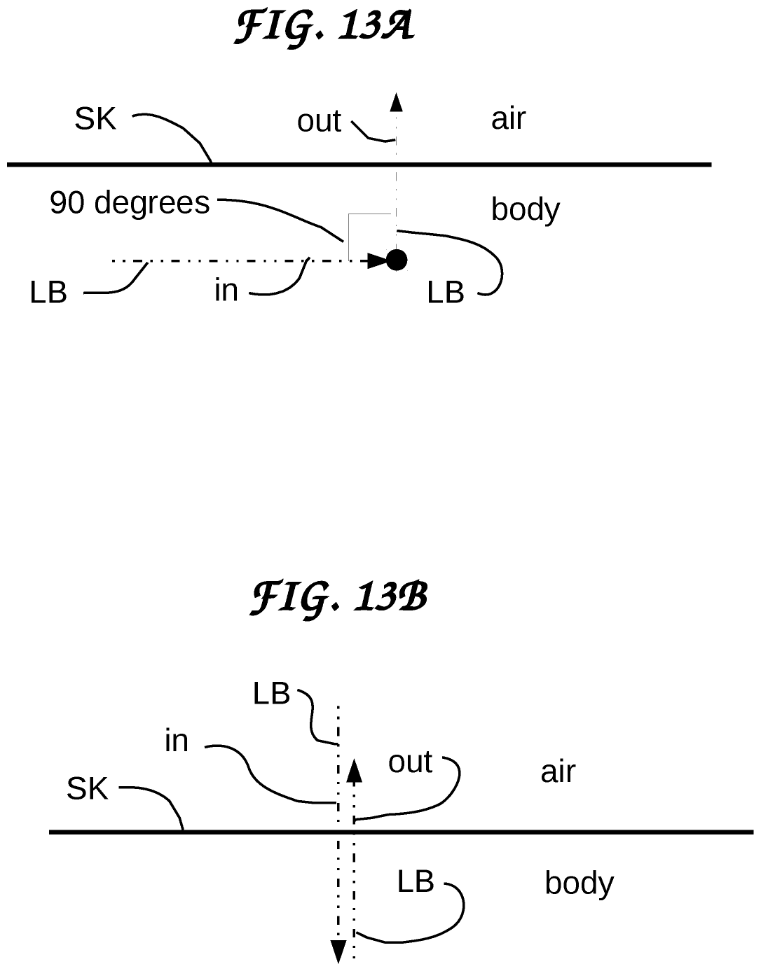

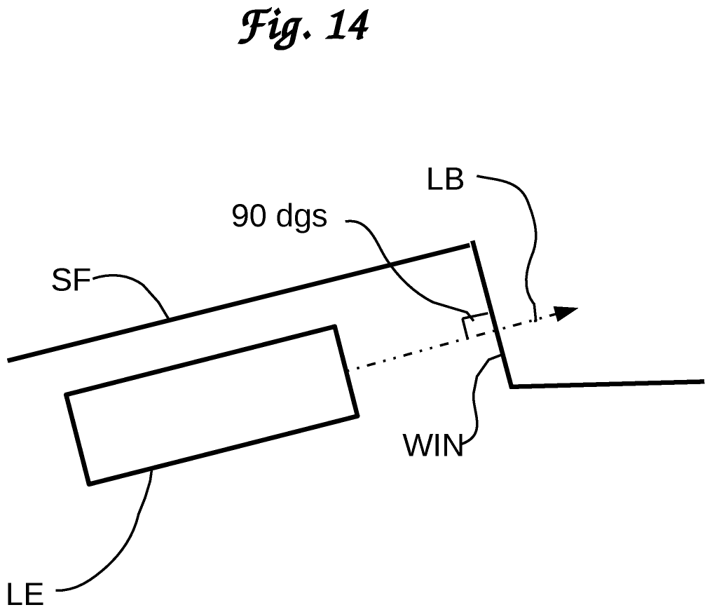

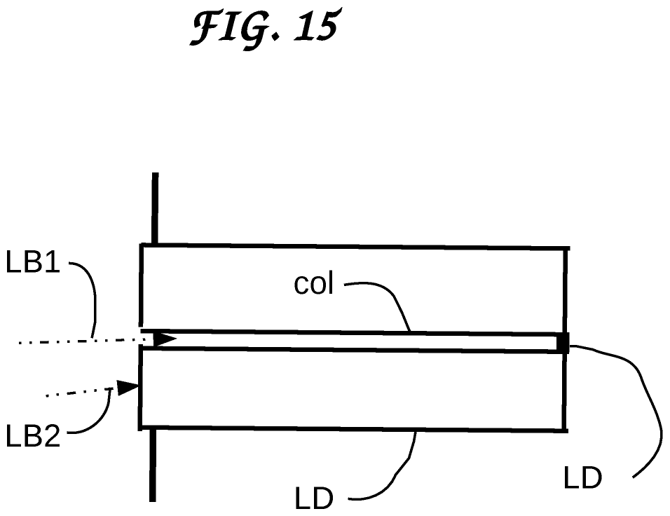

Preferred Embodiment—Figures FIG. 1A and FIG. 1B, and FIG. 3, FIG. 4 and FIG. 6 display some aspects of the preferred embodiment of our invention—for the FitBit variety. These figures should be understood in view of Figure FIG. 7, which is a watch, a normal, ordinary wrist watch, which is similar in shape to the FitBit of our invention. Then figures FIG. 10A, FIG. 10B, FIG. 11A, FIG. 11B, FIG. 9A, FIG. 9B, FIG. 12A, FIG. 12B, FIG. 23, FIG. 13A, FIG. 13B, FIG. 14, FIG. 15 depicts several variations of the main embodiment and some details of the main embodiment.

Figure FIG. 7 shows a wrist watch, which is old art, as the lawyers say, or old stuff in common parlance—let us be real, it is not art at all, only for the damn lawyers! It is shown here only for the reader to identify the equivalent parts on the FitBit of our invention: a box, with either a clock-work mechanical system, for the time-keeping device, or a battery and an electronics circuit, perhaps with added light sources and light detectors and other electrical transducers as well, for the FitBit device, the box being firmly kept at the wrist by either a long strap ST, with some mechanism to adjust its grip on the wrist, as a multiplicity of holes at one of the extremities of it, and a closed loop with a small sticking finger at the other extremity of strap ST. Alternatively, traditional watches and/or some FitBits sports two separate straps ST, each starting at one of the sides of the watch/FitBit at the holding part STH (see figure FIG. 7), in which case one of the straps ST is fitted with the holes at its distal extremity, and the other strap ST is fitted with the closed loop with a small sticking finger at its distal extremity. Our figures are drawn for this latter case of two straps ST, each starting at a holding piece STH on opposing sides of the FitBit, as it is the case of at least most, and I think all traditional wrist watches. It is understood that changing stripe ST to a single longer stripe does not change the nature of the invention. The device of our invention also includes electronics circuits, which are not part of the invention, being old art, well known by electronics engineers, circuits like amplifiers, comparators, time-keeping circuits, oscillators, etc.

Referring now to figure FIG. 6, FIG. 1A and FIG. 1B the reader can see the FitBit of our invention. Figures FIG. 1A and FIG. 1B shown our amazing improved FitBit, firstly in perspective (FIG. 1A) then, at figure FIG. 1B, a cross section of the FitBit of our invention at the plane A-A′, which is shown in the figure FIG. 1A.

Such a design for the amazing FitBit of our invention causes that when the device is firmly attached on the wrist of the wearer, so as to be pressed against the meat, some of the wearer's meat penetrates the volume between the radiation emitter LE (radiation emitter) and the radiation detector LD1 (radiation detector), which are located at the two protruding blocks PB1 and PB2, which is the volume we call the re-entrant cavity. Of course that, as per above observations, the re-entrant cavity may also be a “hole” carved out from the inner surface of the supporting structure SS.

Figures FIG. 10A, FIG. 10B, FIG. 9A, FIG. 11A and FIG. 11B depict variations of the same hardware. The reader is warned that in many of these figures, e.g., figures FIG. 10A, FIG. 10B and FIG. 11A the device surface SF is drawn away from skin SK, which is an exploded view, this having been done only to clearly show the different parts: the mechanical support of the main embodiment and the skin near which the mechanical support is attached. For the main embodiment the device is either a FitBit firmly fixed on the wrist of a person in such a way that the extruding protuberance with the window WIN is pressed against the flesh forcing itself into the flesh, or else there is a re-entrant surface on the external surface of the supporting structure, which is such that, when the FitBit is firmly attached to the wrist the flesh of the wearer penetrates the re-entrant cavity. Either way, the radiation emanating from the FitBit propagates from a transparent window WIN into the skin SK and flesh of the human wearer at an incidence angle of 0 (zero) degrees. The reader is here again reminded that the radiation in the main embodiment is “light”, as red light, infrared light, etc. The width of window WIN is, for the main embodiment, of the order of 400 micrometers, but variations for more and less are possible without changing the invention. This value of 400 micrometers is chosen as a best value because it is enough to allow the light beam LB to exit the FitBit device, and also small enough that the protuberance at WIN is small enough to indent itself in the flesh of the wearer while not to cause discomfort on the wearer. This detail of keeping the window WIN pressed against the flesh of the wrist of the wearer is important for the invention to work, because of the inevitable light beam LB propagation direction change if LB meets the skin of the wearer at any angle other than 0 degrees (perpendicular incidence). This no-deflection characteristic guarantees a known light beam propagation at a desired and known propagation path, almost parallel to the skin and just under the skin, propagating forward at depths ranging from 0.5 mm to 3 mm. This main embodiment is shown at figure FIG. 9A, but FIG. 9A is a blow-out rendering of the situation, with the FitBit device separated from the skin (as opposed to be pressed against the skin), which is done only to clearly show the parts and how they interlock. The reader will notice that the shape of the skin in FIG. 9A follows the shape of the FitBit, exactly because FIG. 9A is a blow-out rendering, in which the skin at the wrist of the wearer assumes the shown shape only because the flesh, being soft as it is, adapts to the shape of the harder surface of the FitBit that is pressed against it—and the reader should keep in mind that the protuberance at window WIN is of the order of 400 micrometers (less than ½ mm), easy to insert itself into the flesh.

Figure FIG. 11B shows the device touching the skin, as it does during normal use. So, repeating with other words as a rampart against lawyers, attorneys, slimes and their likes, figures FIG. 10A, FIG. 10B and FIG. 11A show the device of our invention separated from the skin, this being done only for the purpose of clearly show what is our invention (we did not invent the skin SK . . . :)). Only figure FIG. 11B shows our invention correctly positioned against the skin SK, as it has to be during use, similarly to all FitBit devices.

We want to warn here the reader that in all these figures the penetration of the probing “light” is small if compared with the other dimensions of interest in the drawings. To respect this, and also to simplify the drawings, the dashed lines that indicate the propagation of the “light” beams show a light bean propagating back into the light detector LD as being scattered at the outer surface of the skin, when in reality though some scattering occurs at the surface of the skin, what matters for the invention is scattering that occurred inside the body, as shown in figure FIG. 9B. So the reader is now aware that most figures will indicate a scattering at the very (outer) surface of the skin, when in fact what we mean is scattering that occurred just a little inside, or under the skin as depicted at figure FIG. 9B. Lawyers and other vermins, please do not come to annoy me latter on this, please, please.

Referring now to figure FIG. 15, light detector LD may include a collimator (col) to block “light” propagating from unwanted directions, to enter and being measured by LD. The reason for this is that the image is supposed to be of objects and features at a certain direction with respect to the light detector LD, usually directly in front of LD. Figure FIG. 15 show one example of such a feature, with the light detecting element (LDE) at the end of a collimator (col), which collimator substantially blocks “light” propagating from unwanted directions from reaching the light detecting element (LDE) and being measured as a desired “light”. As shown in figure FIG. 15, a “light” beam LB1, which is propagating along a desirable direction (from directly in front of LD) is capable of reaching the light detecting element (LDE) and be measured, while another “light” beam LB2, which is in such a direction that it would have reached the light detecting element LDE and be measured, is NOT capable of reaching the light detecting element (LDE) at the end of the collimator (col) and is therefore not counted. Throughout this patent application it is understood that a light detector LD may be of this more complex design as shown in this figure FIG. 15, including a collimator, or instead of a simple light detector. Also, such a collimator may be an integral part of the light detector LD, as in figure FIG. 15, or it may be a separate part kept in front of a single detector or an array detector, as a CCD, both possibilities being able to accomplish the same objective of keeping out unwanted “light”.

We will use figure FIG. 10B to describe the preferred embodiment of our invention, but it is understood that many variations are possible, some of these variations shown in the figures that are part of this patent application, and others variations, as per lawyers' approaches, tricks and deceits, are not shown, but intended to be covered by this patent application. The preferred embodiment is a mechanical support box that is held against the skin SK of an animal (usually a human), with a light emitter Light Emitter (LE) that emits a radiation beam at a direction close to, but not completely, parallel to the skin SK of the animal, and with a light detector (LD) a short distance in front of the light beam LB from the point of contact between the light beam LB and the skin sk. In the preferred embodiment the mechanical support that is held against the skin SK is a FitBit device, and the skin SK is at the wrist of the animal. The radiation emitter is preferentially a light emitter LE, which is preferentially a laser, but many other source are possible and compatible with our invention, and the light emitter LE is preferentially along a direction that is almost parallel to the skin SK, say at an angle of 10 degrees with the direction of the skin SK, though this particular value is not the only one that is possible for our invention to work. Of course that it is also possible that the direction of the light emitter LE is any direction, the device including mirrors so positioned that the light beam LB is redirected to a direction substantially parallel to the skin SK of the animal. In general, light beam LB should be almost parallel to the skin SK for the main embodiment, but variations, as described later, are possible, in which the light beam LB is not almost parallel to the skin SK, including the variation of the light beam being normal (perpendicular) to the skin SK. The light detector is preferentially positioned normal to the skin SK, as shown, but normal orientation towards the skin SK of the animal is not the only possibility, other orientations being possible and compatible with our invention. Normal here is used in the mathematical sense, which means perpendicular, as is well known to the readers versed in mathematics, and as defined in the definition section of this patent application. In particular, light detectors that are positioned just out of the incident direction of the incident beam are perfectly compatible with our invention. Such a geometry, such a geometrical positioning of the light emitter LE and light detector LD is achieved with the light emitter LE at one protruding block (PB1) and the light detector LD at the other protruding block (PB2).

The preferred embodiment uses a light emitter LE that emits infrared radiation of wavelengths in the preferred range of 850 nm+−50 nm, that is wavelengths from 800 nm to 900 nm. Any chosen wavelength in this his range of 800 to 900 nm is best because of its higher penetration in animal cells, particularly its lower absorption and scattering cross-sections by the pigment melanin, which is more abundant in humans of darker skin, to the point that existing FitBits fail to work for darker skinned persons (REF 1).

The direction of propagation of the light beam LB is important for this invention to work because if the light beam LB is sent normally (perpendicularly into the skin) then the infrared photons penetrate too deep, into depths where there is less variation in blood irrigation that changes with each heart beat, resulting in that there is only a small variation in the intensity of the infrared back-scattered and the device does not work either, even if the light beam LB penetrate beyond the melanin layer. Note that my invention does not depend on this theory of the depth of penetration to be correct, but only in the experimental results from the inventors' experiments. Moreover, though the invention itself came from pure cerebration, the actual confirmation by experiment is all that matters for the patent application, not the theory of why it works, not the process of cerebration that brought the solution of the problem to the attention of the inventor.

When the light beam LB is propagating parallel to, and just below the skin SK, then the infra-red photons are mostly in a path where the change in blood irrigation suffers maximum variation with each heart beat. This is so even if some photons happen to penetrate deeper below the skin, due to both the initial beam angular divergence and also due to forward scattering. It then follows, from this geometrical configuration and maximum interaction that matters for the working of this invention, that the variation of scattered infra-red photons is larger than other paths of photon propagation, particularly larger than photons propagating perpendicularly into the and below the skin sk. Another advantage for using this path of propagation which is almost parallel to, and just below the skin SK, is that photons propagating along such path needs to be scattered by 90 degrees to be measured by the light detector LD, as opposed to be scattered by 180 degrees (completely backward), as is the case with most existing FitBits. It happens that the scattering cross section as a function of the angle of scattering is generally a monotonically decreasing function of the scattering angle, which then implies as per figure FIG. 13A and FIG. 13B, that there is less photons to measure when the illumination is perpendicular to the skin SK (180 degrees scattering, figure FIG. 13B) than when the illumination is just below the skin SK, almost parallel and just under the skin SK (90 degrees scattering figure FIG. 13A), as is the configuration of our invention. According to the measurements taken by the inventor, such a parallel propagating light beam LB produces maximum variation in the total energy of back-scattered photons. We warn the reader that we are here using the technical language used in physics, because in common language this would be said “ . . . produces maximum variation in the total energy of the sideways (90 degrees) scattered photons.”. In physics, all photons scattered at an angle larger than 90 degrees with the directions of propagations are called back-scattered—though this statement is misleading when taken literally in common English. This physics wording happens because we physicists call anything that is scattered into the forward hemisphere (less than 90 degrees deviation with the initial beam) forward scattering, and accordingly, anything that is scattered into the back hemisphere (more than 90 degrees deviation with the initial beam) back-scattering.

The position of the light emitter LE is fixed with respect to the skin SK of the user by the surface of the mechanical support, which, in the preferred embodiment is a FitBit-type device, as shown at figures FIG. 3, FIG. 4 and many others.

The depth of the indentation on the FitBit surface, which is the width of the window WIN, is typically of the order of a fraction of a millimeter to a millimeter or two, and the corners of the indentation or almost-perpendicular surface are rounded to prevent scratching the skin of the FitBit user. Window WIN is, on the preferred embodiment of our invention, perpendicular to the direction of the light beam LB. This is another important feature to prevent causing discomfort on the FitBit wearer.

Light detector LD receives light scattered from the region under the skin of the wearer. The intensity of this light scattered into the light detector LD varies with the amount of blood in the region from where the light beam LB is scattered, causing a periodic variation of the light intensity detected (measured) by light detector LD. This periodic variation of the light intensity follows the heart beatings. This periodic variation can be measured, converted by an ordinary ADC (analog-to-digital converter), then the digital result can be transferred to a microcontroller and counted over any convenient time period, say, 15 seconds, or 30 seconds or any other time. After normalizing the number of variations to 60 seconds, this normalized counting is the number of heart beatings per minute (one minute is 60 seconds). Any ordinary microprocessor, which is already part of the existing FitBits, can do this process of “watching” the periodic variation of the light intensity at the light detector LD.

### EXAMPLES OF INTENDED USES

One example of intended use is to monitor the heart beating rate of humans wearing FitBit-type devices intended to acquire data about their physical activity, either for health reasons or for the purpose of improving their physical performance or even just to show-off.

Another intended use of the device and method of our invention is to measure the oxygen saturation/concentration in blood.

Another intended use of the device and method of our invention is to improve on the data gathering for hospitals and similar health facilities, that may need to collect information on the oxygen concentration/saturation for health decisions. Our device, as similar, though not as good, oximeters, produce a number, which goes well with the current trend of “evidence-based” diagnostics, which deflects any possible lawsuits by the damn lawyers.

Another intended use of the device of our invention is to buy a home for the inventors and to pay-off Diane's farm.

### DETAILED DESCRIPTION—OPERATION OF INVENTION

This first paragraphs of the section “detailed description” is from the mother patent, which is for FitBits; the part that matters for this current continuation-in-part patent application, the extention to oximeters, is at the section “conclusion, ramifications, and scope of invention”.

I am adding a theoretical analysis of the invention because it helps the reader to better understand the invention and also to reproduce it. It is my view that a clear understanding of the structure that will be described in the sequel is only complete with an understanding of the reasoning underlying it, as opposed of a simple and magic physical description of the device—amazing as it is!

The method of our amazing invention is to direct the energy probing beam to a propagating path just under the skin, for both the FitBit and the oxymeter wearer, that is, propagating generally parallel to the skin and just under the skin, say, from 500 micrometers (0.5 mm) to 10 millimeters under the skin, preferably 500+−200 micrometers under the skin, the 10 millimeters being an upper limit that is entering here only because of the damn lawyers. The probing beam is preferably either visible light, or even better, what is known as deep red (red near the end of the visible red, around 700 nm), or even better, near infrared radiation, preferably near 850 nm. The reasoning for this is discussed and explained in the theoretical analysis below, it has to do with the smaller absorption by flesh of photons of these wavelengths in the near infra red.

Our invention operates on the differential cross section between blood and other animal cells, particularly between blood and flesh. This statement, which is written in physics language can be re-stated in normal English as “Our invention operates on differences of scattering properties, or probabilities, between blood and other cells of animals”.

We note here that this detection/recognition using differential scattering cross-section is no difference between all our vision system that we use all the time for all things, and image detection hardware used by the hardware and the computer of our invention. It is what we do all the time from observing a painting on a museum or to read the letters in this funny written patent application, written by an unconventional retired professor of physics. We decide that something is a leaf and not a flower both using the form of them and also using their color, i.e., using the differences in scattering of each for different colors. For example, most leaves scatters green light, some of which enters our eyes, absorbing the rest (red, yellow, etc.), while most flowers scatters a particular color, say red (as a red rose does), some of which red eventually enters our eyes, absorbing the other colors (yellow, green, etc.). In the case of our invention, as long as blood has a different scattering cross section (this is technical language, meaning probability of scattering, or power to scatter, or capacity of scattering), then the scattered “light”, which is preferentially infrared “light” for the main embodiment of our invention, will show the difference, as detected by a camera, and later measured by a computer. It is this simple, no big deal! . . .

The preferred embodiment of our invention uses near infrared radiation, with wavelengths in the window from 800 nm to 900 nm, because this range of wavelengths penetrates more in flesh, being, therefore able to probe deeper than other “colors”. For example, many FitBits use green light, which is so much absorbed by melanin that it cannot penetrate (and come back out after scattering!) enough to show any change in absorption and scattering cause by any change in blood irrigation due to heart beatings. It is a known fact that these FitBits that use green light works from poorly to not at all for darker skinned people. But keep in mind the particular detail that allows the green-light FitBits to work on fair-skinned people, as discussed further down.

Besides using infrared “light”, our invention uses a “light” beam that propagates parallel to and just below the skin, as opposed to propagate into the body, especially as opposed to propagating perpendicularly to the skin at the penetration point, or, in other words, that propagates parallel to and just under the skin, as opposed to propagate perpendicularly to the skin. The reason for this is different than the depth of penetration. Our invention uses a beam propagating parallel to and just under the skin SK, because of two independent reasons. Firstly because it is just under the skin that occurs the largest change in blood irrigation with each heart beat, or with each increase in blood pressure at each systole (systole means the higher blood pressure, or the heart contraction). To say it in a different way, our invention uses a “light” beam parallel and under the skin because it is there, just under the skin, that occurs the largest change in blood irrigation with each heart pumping, and consequently there is the largest change of the measured quantity: the amount or intensity of scattered “light”. Secondly, our invention uses a “light” beam propagating parallel to and just below the skin because with this path of propagation the scattered “light” has to suffer a scattering event of between 0 (zero) to 90 degrees to be measured by a light detector LD out of the body, instead of a 180 degrees scattering event, as it is the case for a light beam propagating perpendicular into the body, as used by most existing poorly designed FitBits. This makes a sizable difference for the measurement, because in just about all cases, and it is true in this case, the scattering cross section (meaning, the scattering probability) is much larger for a 0-to-90 degrees scattering than for a 180 degrees scattering, so the geometric arrangement of our invention causes that more “light” reaches the light detector LD than the existing FitBits that illuminate the body of interest with perpendicular light then receives 180 degrees scattered light! It is this simple . . . . This is illustrated in figure FIG. 13A for a 90 degrees scattering and FIG. 13B for a 180 degrees scattering. This latter, FIG. 13B, 180 degrees scattering is what many would call back scattering in normal, ordinary, common English.

### DETAILED DESCRIPTION—DESCRIPTION AND OPERATION OF ALTERNATIVE EMBODIMENTS

Figure FIG. 14 shows a detail of the mutual positioning of the light emitter LE, the light beam LB and the window WIN, and the preferred angle between the light beam LB and the window WIN, which is, for the main embodiment of this invention, preferably 90 degrees, as shown. The reason for this is to prevent multiple reflections at the entrance and exit surfaces of the window WIN.

Variation of the Main Embodiment Applied to Oximeters

One interesting variation of the main embodiment of the mother patent (FitBit), is the application of the same principles of the FitBit to measure heart rates, to the related application of measurement of oxygen concentration/saturation in blood (oximeter), from now on referred simply as oxygen saturation. Both cases require measurement of blood properties with spectroscopy, emitting “light” into the body, then measuring either the scattered or the non-scattered “light” back out, after this “light” has suffered two passages through the skin (in-and-out), perhaps being partly absorbed by melanin (REF 1).

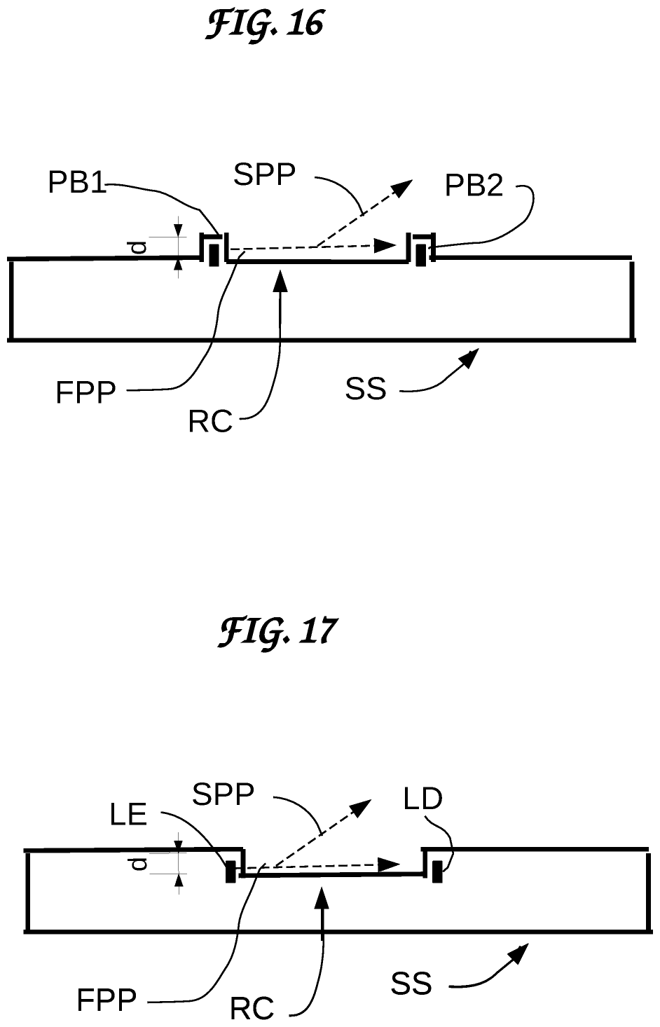

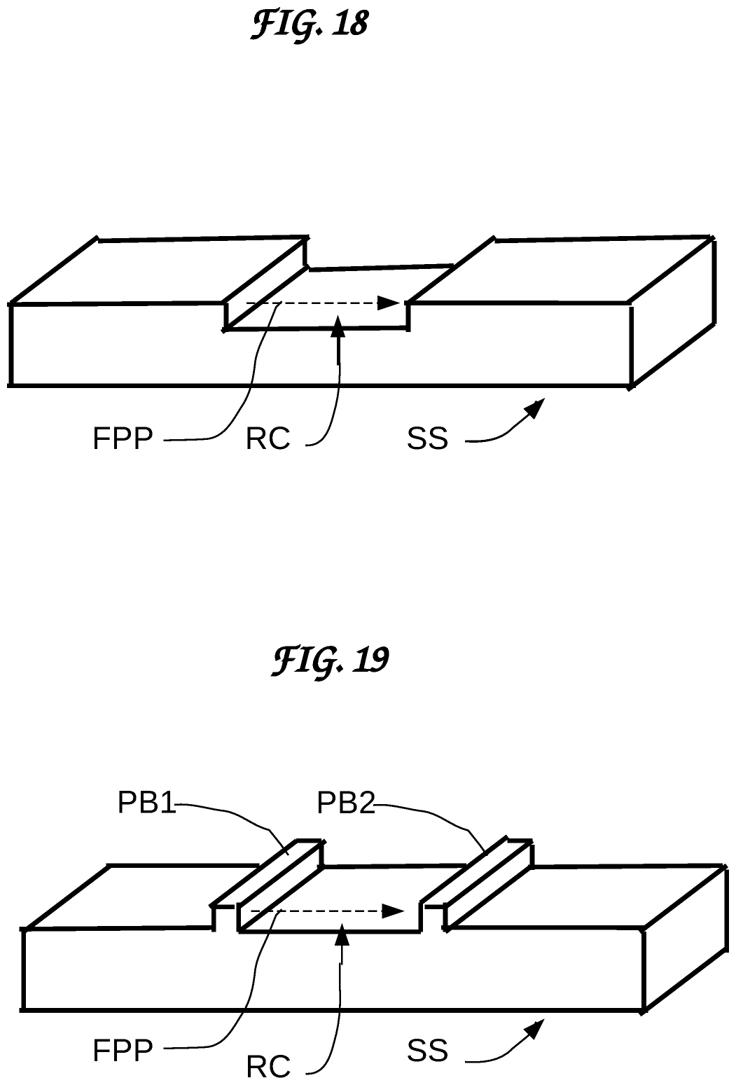

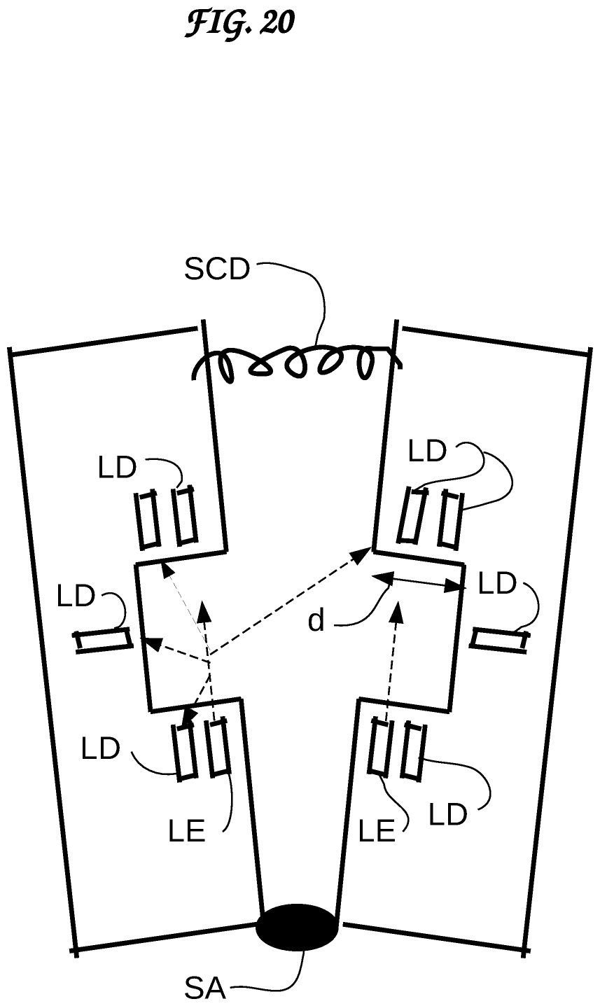

For a better understanding of the device, and how it works, we proceed describing a simple version of the device of our invention, which is good to highlight the principles of operation, which we will be followed by the section “main embodiment” where we will describe a real proposed main embodiment for the invention. This simpler device may be thought as a theoretical description, because it highlight the principles of operation detached from any practical implementation. FIGS. 16 and 17 display the main elements of the invention: a supporting structure SS, a re-entrant cavity RC, with depth d, one (or more) light emitters LE, one (or more) light detectors LD, a first propagation path (FPP) and a second propagation path (SPP), this second propagation path not shown for simplification. The first propagation path is the direction, or line of propagation, of the photons emitted by the light emitter LE, while the second propagation path is any of the directions, or line of propagation, of the photons that have suffered at least one scattering event, therefore changing their direction of propagation away from the first propagation path. The re-entrant cavity RC is the same as the RfitBit, which was the specific name for the re-entrant cavity for the FitBit, used, for example at FIG. 8. The re-entrant cavity RC is there to force a part of the flesh of the animal to which the supporting structure SS is attached to penetrate the re-entrant cavity, this being so because it allows for a known volume of flesh and blood to be probed by light beams, which volume contains part of the blood vessels located just below the skin of the animal and also because, due to the location and direction of light propagation path FPP, the illuminating light beam is approximately parallel to the skin. This, in turn, is so because the blood vessels just below the skin are closely connected to the heart than many of the blood vessels that are deeper under the skin, which is important for the devices, both the FitBit and the oximeter. These two side views of the general features of our invention is repeated at FIGS. 18 and 19, which show the same thing as the side view, but in perspective. Other figures show the same with more details.

The re-entrant cavity RC may be made as a cavity carved out into the surface of the supporting structure SS which faces the skin of the animal, as shown at FIGS. 18. 19 and others. Also, as explained above, instead of a re-entrant cavity RC there may be two protruding blocks PB, which defines a space, or a hole, or a cavity between them, which functions the same as the re-entrant cavity RC. Both the hole and the protuberances are equivalent for our invention.

Detailed explanation of a particular embodiment.

This variation to the mother patent (FitBit), applied to oximeters, may be implemented with a variety of supporting structures, all of which are small modifications/variations of the same basic design. Accordingly, we are going to use one particular incarnation of the main embodiment of this daughter application, which uses a supporting structure similar to a cloth pin, of the type used to hold in place clothes to dry on a cloth-line. We chose this particular incarnation because it is one of the most commonly used types of oximeters today, if not the most common. It is the cloth-pin-type device that the health practitioner clips to our index finger as soon as we enter the facility, since the COVID-19 invasion, approximately March 2019 in the Unites States.

What is known as oxygen saturation, is measured with a comparison of the readings at two different wavelengths, from which the amount of oxygen with a particular molecular structure in the blood, within the probed volume, can be determined (REF 2). The radiation measured can be either transmitted radiation or scattered radiation, both types are possible and exist, The words “probed volume” above are crucial for out invention, which may be described as a volume which is selected for measurements by the device, that causes that the blood volume probed, or measured, is just under the skin, as opposed to probe deep under the skin. There are reasons for this, as described before and after here, which are related to the blood circulation and distribution, starting from the heart's left ventricle, to the main arteries, arterioles, capillaries, venules, main veins then back to the heart's right atrium.

The two wavelengths are usually red, or deep-red, and infrared, or near-infrared. More precisely, 700 nm (red), let us call this wavelength lambda1, and 850-900 nm (near infrared), let us call this wavelength lambda2. These two wavelengths lambda1 and lambda2 are usually along the same direction of propagation, or along the same line of propagation, or along the same propagation path, which we will call first propagation path FPP, is the set formed by the two photon beams at lambda1 and lambda2 we will call first beam. Consequently the first beam is a compound beam of photons at two wavelengths lambda1 and lambda2. For the damn lawyers out there, these are just two examples for wavelengths, the invention can work with other wavelengths as well, these two wavelengths being just two of the most used currently and being here as examples, not the only possibilities, but other wavelengths are possible as well. There are devices on with 5 different wavelengths. Don't come to me later, you damn lawyer, representing a client that is marketing an oximeter using 699 nm and 849 nm, pretending that these are outside the scope of my invention! These two wavelengths are selected by the differential absorption and/or scattering cross section (probability) of oxygenated hemoglobin, as compared with the less oxygenated form of hemoglobin.

Both the FitBit and the oximeter are subjected to the same problems of melanin absorption, when used on darker-skinned persons (REF 1). Melanin absorbs the incident light, resulting that there is at least less photons, often not enough photons to make the desired measurement, or even none at all. Both devices work better on fair-skinned persons. This problem is well documented in the literature, e.g., John R Feiner, John W Severinghaus, Philip E Bickler, “Dark skin decreases the accuracy of pulse oximeters at low oxygen saturation: the effects of oximeter probe type and gender” Anesth Analg December 2007, Vol 105, Number 6 (suppl), pg 518 ff., and Sjoding, Michael W. M., D. Dickson, Robert P. M.D., Iwashyna, Theodore J. M.D., Ph.D., Gay, Steven E. M.D., Valley, Thomas S. M.D., “Racial Bias in Pulse Oximetry Measurement”, New Engl. J. Med. V 383 issue 25 pages 2477-2478.

From now on, when referring to this wonderful oximeter of our invention, it will be understood that the light emitter LE is made of the two beams at wavelengths lambda1 and lambda2, as described above, which are along the same line of propagation or first propagation path FPP. This beam composed of the two separate wavelengths will be either two light emitters, perhaps encapsulated in the same container, perhaps separate emitters with the necessary optics to cause that their radiation beams are along the direction we call the first propagation path FPP, emitting light (radiation) on two different wavelengths, which normally are deep red and near infrared, usually at a first wavelength lambda1 equal to 700 nm and a second wavelength lambda2 within the range 850 to 900 nm, but may be other wavelengths or colors as well. Two wavelengths are necessary. So again, for you damn lawyers out there, from now on, on all instances of the oximeter variation, when we refer to light emitter LE it will be understood that these are two light emitters LE, say LE1 referring to deep red, lambda1, say, 700 nm, and LE2 referring to near infrared, lambda2, say 850-900 nm, and that these two light emitters may be one single package, or even one single device capable of emitting these two wavelengths.

The oximeter of our invention uses the same method of keeping the “light”, or radiation, propagating just near, and under, the skin surface, as described in the specification of the mother applications for the FitBit. In both cases this is so in order to cause that the “light” path is confined to volumes inside which the circulating blood is near the skin and near the original pumping heart than the blood that is deeper under the skin. The probing light beam(s) is (are) forced to be just under and not deep under the skin by arranging the supporting structure to direct the probing light beam LE inside the re-entrant cavity of our invention.

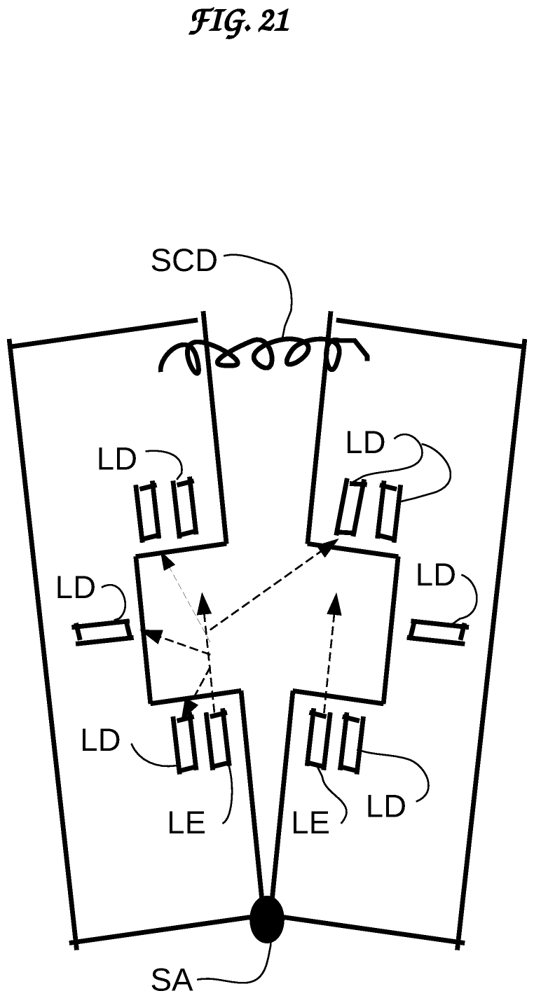

One embodiment, among others, of the oximeter, extension-variation of our beautiful FitBit device, is a device to measure the oxygen concentration/saturation, which, for the main embodiment, built within a supporting structure that is in the shape of a cloth pin, of the style used to keep clothes to dry hanging on a rope on the sun. This is the most commonly shape of the existing oximeters currently in use. It is shaped and sized as to attach to a finger or an ear lobe, etc. of a human being, or other animal. FIGS. 20 and 21 depict two examples of this. The oximeter attached to the tip of the finger may be the most common shape of the existing oximeters, but this may change without changing the nature of our wonderful invention. The actual shape of the supporting structure can vary, without changing the nature of the device. Our invention is the addition of the re-entrant cavity to the supporting structure SS, which is, for the main embodiment, a device on the general shape of a cloth-pin. The re-entrant cavity RC exists for the purpose of keeping the probing light beam propagating approximately parallel to and just below the skin of the animal.

In FIGS. 20 and 21, the reader can see that when the supporting structure is clipped onto the finger of an animal, or an earlobe of an animal, or other part of an animal, the necessary elements, one or more “light” emitter LE and one or more “light” detector LD (or radiation emitter and radiation detector) are so positioned that the light emitted by the light emitter LE, propagates inside the volume of the re-entrant cavity RC, inside which a part of the flesh of the animal is pressed to penetrate. The depth d of the re-entrant cavity is small to facilitate the flesh of the animal to be forced into the re-entrant cavity RC. The depth d controls the distance, under the skin, which is probed by the light emitted by LE, which is typically from 0.1 mm to 100 mm, but may be a smaller value as well, e.g., from 0.1 mm to 10 mm, or even smaller, as 0.1 mm to 5 mm, or to 3 mm. The actual numerical value of the depth d is not part of the invention. A consequence of the depth d of the re-entrant cavity RC being small, is that it is only the flesh near the skin that is capable of being forced into the re-entrant cavity RC, which is what we want!, to probe blood just under the skin! This being so, the oximeter is virtually the same as the FitBit of the mother invention. FIG. 3, which is for the FitBit, shows this penetration of the flesh into the re-entrant cavity, the same as for this oximeter variation. Of course that the FitBit with a re-entrant cavity RC may also include an oximeter—as long as they give us some dough.

Such a device has two variations. Variation CPT (ClothesPinTransmission) is a clothespin with light emitter devices LE1 and LE2, usually referred in common as LE, which propagate along a first propagation path FPP, and a light (or radiation) detector LD aligned with the first propagation path FPP, detecting photons that have not suffered any scattering episode and therefore reach the light detector LD along the same direction as the photons were emitted by LE. In this variation, the propagation path of the photon that reaches the light detector LD is the same as the first propagation path, because there is no scattering, and the device working on the transmission mode. There may exist a collimator as well. Such variation CPT is used on the transmission mode, and it measures “light” that has suffered no scattering,

Another variation of the same device, is the variation CPS (ClothesPinScattering), which is the same clothespin CP with the light emitters LE1 and LE2, the set that we here call LE, and light detector LD out of alignment. For the CPS the emitted light is along a propagation path which we call first propagation path FPP, along which path the photon (the particle of light that is propagating along this first propagation path) suffers a scattering, the result of which being that the photon then propagates along a second, different radiation direction, which we will call second propagation path SPP.

The re-entrant cavity RC of our invention may be the result of a hole on the surface of the supporting structure SS, or it may be the result of the light emitter LE and light detector LD be inside separate protruding blocks PB1 and PB2, which then create a space in between them. The former case is shown at FIG. 17, and the latter case is shown at FIG. 16. They are topologically equivalent, as they both create a space where the flesh of the animal penetrates, causing that the propagating light beam stays just under the skin SK of the animal, and propagating approximately parallel to this skin SK.

Another possibility is a re-entrant cavity similar to the one used with the FitBit. This variation device for oximeter may easily be incorporated into a FitBit device.

The re-entrant cavities may be created either by a re-entrant slice or hole onto the inner surface of the supporting structure, as the clothpin CP, or the FitBit, or a blood pressure cuff, which is the surface that touches the skin of the finger (or whatever other part of the body), or the re-entrant cavity may be created by two protuberances PB or extrusions EXT, extending out from the inner surface of the clothpin CP, or the FitBit, or the blood pressure cuff, etc., which generate an re-entrant cavity between them. Either way is good, technically equivalent, I do not care, as long as I get the money for them. Incidentally, I will not get money for the invention applied to the exploited lands of South America, Africa, etc., but only from the exploiters from USA and Europe, and the money will be used to create and maintain research institutes, libraries, health clinics, etc . . . , for the benefit of the exploited people.

So, repeating it in other words, the re-entrant cavity RC may be made either as a cavity on the inner surface of the supporting structure, or there may exist instead two optional extrusions which we call protuberances PB, which create the equivalent of a re-entrant cavity between them; such two optional extrusions or protuberances PB are therefore already covered by the re-entrant cavity but we mention them protuberances PB because of the damn lawyers who will latter come with their non-sensical absurdities.

Another embodiment of the oximeter extension-variation of out wonderful FitBit invention, which already gave us one issued and one allowed patent (:)) is the use of the ordinary cuff used for blood pressure measurements as supporting structure for the light emitter LE and the light detector LD, the rest being the same. We call this extension-variation as the BP oximeter, or BPO. Such variations either would use the device on the scattering mode, which we call the BPOS, or on the transmission mode, which we call the BPOT. The former case, the scattering mode, has the light emitter LE and the light detector LD just laid on the surface of the cuff, in which case they are not aligned, as they need not be for the scattering mode. For the transmission mode, which we call Blood Pressure Oximetry Transmission (BPOT), there exists a solid supporting structure, preferably small, which may be as small as 5 mm, with two protruding blocks, a protruding block PB1, where a light emitter LE, emitting LE1 and LE2, is fixed and emitting light approximately parallel to and just under the skin SK of the animal, and a light detector LD is fixed on the opposite protruding block PB2, which creates a re-entrant cavity between the two protruding blocks. For this variation of the BloodPressure BP variation, the light emitter LE is aligned with the light detector LD and the device works on the transmission mode described for the beautiful FitBit device of our invention.

Naturally that the oximeter could also be used as part of the FitBit itself! In this case most of the physical difference is the use of two wavelengths (“colors”), LE1 and LE2, then the extra parts on the software to calculate the oxygen saturation from the two measurements.

Another variation is a cuff-type of support device that is worn on the neck of a person, or other animal, the rest being similar to the blood pressure BP variation described above.

Another variation is a cuff-type of support device that is worn on the leg of a person, or other animal, the rest being similar to the blood pressure BP variation described above.

Another variation is a free-standing device FS, which can be a flexible type of support or a rigid type support, which would be positioned by hand on the desired location, and pressed on the skin of the person, or animal under examination, causing the flesh to penetrate the re-entrant cavity and the rest is the same.

### Conclusion, Ramifications, and Scope of Invention

It is worth to mention that another class of devices, to form images using infrared (mostly) in transmission and forward scattering is besieged by the same problem as the propagation of the infrared from the FitBit into the wrist, and both require similar solutions.

Thus the reader will see that the illuminator of our amazing invention that so much improves the data collection for FitBit-type devices provides a highly reliable, lightweight, yet economical device that can be used by persons of almost any age and skill. In particular the illuminator of our invention contributes for the device, FitBit or any of its variations, to be usable for individuals of darker skin complexion.

While my above and below description contains many specificities, these should not be construed as limitations on the scope of the invention, but rather as an exemplification of one preferred embodiment of the invention (words borrowed from a lawyer . . . ). Many other variations are possible. For example, the light source may be of other colors, as visible red, which, though being more absorbed by the skin, including melanin, is still less absorbed than other colors of shorter wavelengths, as green, etc. The shape of the light emitted by the light source may also be altered in many ways. For example, the light beam may be spread along one direction only by a cylindrical lens (also known as astigmatic lens and non-spherical lens), capable of illuminating the body with a light “sheet” so to say, or a light distributed spread along a sheet parallel to, and slightly under the skin of the individual. Such a light distribution has the advantage of producing scattering from a wider area, therefore reaching more capillaries that are capable of producing the required optical signal when compared with a beam that is narrow, or laser-like, which by necessity probes a smaller number of capillaries than the sheet-like light beam. When using such a light distribution, the plane that defines the light distribution should preferably be approximately parallel to the skin.

Accordingly, the scope of the invention should be determined by the embodiment(s) illustrated, by the appended claims and the figures, and any lawyer's future confusing talk and their legal and illegal equivalents (copied from a lawyer, with variations).

### SEQUENCE LISTING

Not applicable.

### REFERENCES

Ref 1=https://www.healthline.com/health/pulse-oximetry#whats-next accessed Nov. 11, 2022, 3:20 pm
REF 2=https://en.wikipedia.org/wiki/Pulse_oximetry accessed Nov. 11, 2022, 3:35 pm
Pulse oximetry is a noninvasive method for monitoring a person's oxygen saturation. Peripheral oxygen saturation (SpO2) readings are typically within 2% accuracy (within 4% accuracy in 95% of cases) of the more accurate (and invasive) reading of arterial oxygen saturation (SaO2) from arterial blood gas analysis.[1] But the two are correlated well enough that the safe, convenient, noninvasive, inexpensive pulse oximetry method is valuable for measuring oxygen saturation in clinical use.[citation needed]
The most common approach is transmissive pulse oximetry. In this approach, a sensor device is placed on a thin part of the patient's body, usually a fingertip or earlobe, or an infant's foot. Fingertips and earlobes have higher blood flow rates than other tissues, which facilitates heat transfer.[1] The device passes two wavelengths of light through the body part to a photodetector. It measures the changing absorbance at each of the wavelengths, allowing it to determine the absorbances due to the pulsing arterial blood alone, excluding venous blood, skin, bone, muscle, fat, and (in most cases) nail polish.[2]
Reflectance pulse oximetry is a less common alternative to transmissive pulse oximetry. This method does not require a thin section of the person's body and is therefore well suited to a universal application such as the feet, forehead, and chest, but it also has some limitations. Vasodilation and pooling of venous blood in the head due to compromised venous return to the heart can cause a combination of arterial and venous pulsations in the forehead region and lead to spurious SpO2 results. Such conditions occur while undergoing anesthesia with endotracheal intubation and mechanical ventilation or in patients in the Trendelenburg position
A pulse oximeter is a medical device that indirectly monitors the oxygen saturation of a patient's blood (as opposed to measuring oxygen saturation directly through a blood sample) and changes in blood volume in the skin, producing a photoplethysmogram that may be further processed into other measurements. [4] The pulse oximeter may be incorporated into a multiparameter patient monitor. Most monitors also display the pulse rate. Portable, battery-operated pulse oximeters are also available for transport or home blood-oxygen monitoring.[5]

1. An apparatus for gathering information on a circulating blood of animals, including humans, said animals with a body, a skin, and a first surface of said skin of said animal, said apparatus comprising:
a) a holding and containing device adapted to hold, contain and maintain in fixed position against said skin of said animal, a plurality of elements, where said plurality of elements are at least one or more from a set of elements composed of at least one radiation emitting device and at least one radiation detecting device, such that:
a1) each said radiation emitting device, emitting radiation along a first propagation path characteristic to each of said at least one radiation emitting devices, and capable of emitting said radiation of one or more wavelengths,
a2) each said radiation detecting device receiving said radiation from one or more of said radiation emitting devices, along a second propagation path, that is different than said first propagation path characteristic of each of said at least one radiation emitting device, and, furthermore, capable of detecting at least part of said radiation of one or more said wavelengths,
a3) each said radiation detecting device receiving said radiation from one or more of said radiation emitting devices, is capable of measuring a intensity of said radiation along said second propagation path, originally emitted by said radiation emitting devices and which suffered one or more scattering events,
b) where said first propagation path of said radiation emitted by said at least one radiation emitting device is different than said second propagation path to said at least one radiation detecting device that detects radiation emitted by said radiation emitting device,
c) where said first propagation path of said radiation emitted by said radiation emitting device, said radiation that is propagating inside said animal, is a known said radiation beam propagating at a desired and known first propagation path, just under said first surface of said skin of said animal, propagating forward at depths ranging from 0.5 mm to 10 mm below said first surface of said skin SK of said animal,
d) where said holding and containing device is formed with a re-entrant cavity such that, when said holding and containing device is fixed in position, a flesh of said animal, including human , is pressed to fill in a space of said re-entrant cavity.

2. The apparatus of claim 1 were said radiation is either infrared radiation or deep red radiation.

3. The apparatus of claim 1 where said holding and containing device is kept in fixed position against said skin of said animal, at some part of a first surface of said skin of said animal, including human, by a wrapping device adapted to keep said holding and containing device in fixed position against said skin of said animal.

4. The apparatus of claim 1 where said first propagation path inside said body of said animal of said radiation emitted by said radiation emitting device is just under said skin of said animal from 500 micrometers (0.5 mm) to 5 millimeters under said skin.

5. The apparatus of claim 1 where said first propagation path of said radiation emitted by said radiation emitting device, that is propagating inside said animal, is along a direction that makes an angle less than 30 degrees with said first surface of said skin SK of said animal.

6. The apparatus of claim 1 where said radiation detecting device is capable of detecting and measuring said intensity of said radiation that has suffered one or more scattering events inside said body of said animal, and propagates after said scattering event along said second propagation path that makes an angle with said first propagation path of said radiation emitted by said radiation emitting device, said angle being between 1 degree and 180 degrees.

7. The apparatus of claim 1, where said re-entrant cavity is created on said holding and containing device by a cavity on said holding and containing device, said cavity existing on a side of said holding and containing device, that is facing said flesh of said animal.

8. The apparatus of claim 1, where said re-entrant cavity is created on said holding and containing device by a pair of protruding elements on said holding and containing device, on a side of said holding and containing device that is facing said flesh of said animal.

9. The apparatus of claim 1, where said re-entrant cavity is created on said holding and containing device by a single protruding element on said holding and containing device, on a side of said holding and containing device that is facing said flesh of said animal.

10. The apparatus of claim 1 where said information on said circulating blood of said animals, including humans, is a measurement of said intensity of said radiation beam at two different wavelengths.

11. The apparatus of claim 1 where said information on said circulating blood of said animals, including humans, is a measurement of a volumetric change per unit of volume on said circulating blood of said animals, including humans.

12. The apparatus of claim 1 where a second surface of said holding and containing device is a flat second surface while said radiation emitted by said radiation emitter LE is positioned at a first shallow angle with said second surface of said holding and containing device and with said skin of said animal, and said radiation detector LD is at a second shallow angle with said second surface of said holding and containing device and with said skin of said animal.

13. The apparatus of claim 1 where a second surface of said holding and containing device is a flat second surface, while said radiation emitted by said radiation emitter LE is positioned at a first shallow angle with said second surface of said holding and containing device and with said skin of said animal, and said radiation detector LD is at a position making a second angle larger than 15 degrees and smaller than 165 degrees with said second surface of said holding and containing device and with said skin of said animal.

14. A method for gathering information on a circulating blood of an animal, including humans , where said animal has a body, a skin and said skin has a first surface, wherein said method comprises the following steps:
1) providing a holding and containing device kept in fixed position with respect to said skin of said animal, including humans adapted to hold, contain and maintain in fixed position one or more elements, where said elements are one or more elements from one or more groups of elements where said groups of elements are one or more groups from the set of groups: one radiation emitting group and one radiation detecting group, such that:
1a) one or more radiation emitting devices, which is a member of said group of radiation emitting group, adapted to emitting a radiation at one or more wavelengths, each said radiation emitting device emitting said radiation at one or more wavelengths along a first propagation path, which is characteristic of each said radiation emitting device,
1b) at least one said radiation detection device, which is a member of said radiation detection group, adapted to detecting said radiation at said first wavelength lambda1 and at said second wavelength lambda2, each said radiation detection device detecting said radiation along a second propagation path and within an acceptance angle, which are characteristic of each said radiation detection device,
2) where each of said at least one said radiation detection device is positioned so as to detect radiation emitted by said radiation emitting device that have suffered one or more than one scattering events inside said animal, and after said one or more scattering events, propagates along said second propagation path that makes an angle larger than zero degrees and less then 180 degrees with said first propagation path of said radiation emitted by said radiation emitting device,
3) fixing said one or more radiation emitting device at such locations with respect to said holding and containing device, that said radiation emitted by said radiation emitting device propagates inside said animal, including humans, along said known first propagation path, just below said first surface of said skin SK of said animal, located at a closest proximity to said known first propagation path and at a depth of less than 1 cm from said first surface of said skin of said animal located at said closest proximity to said known first propagation path of said radiation under said skin of said animal,
4) where said holding and containing device is formed with a re-entrant cavity such that, when said holding and containing device is fixed in position, a flesh of said animal, including human, is pressed to fill in a space of said re-entrant cavity.

15. The method of claim 14 with a further wrapping device adapted to keeping said holding and containing device in fixed position with respect to said skin of said animal.

16. The method of claim 14 where said radiation emitting device emitting said radiation at one or more said wavelengths is either a single device capable of emitting said radiation at a first wavelength lambda1 and at a second wavelength lambda2, or is a combination of two separate radiation emitting devices, capable of emitting said radiation at said first wavelength lambda1 and at said second wavelength lambda2 each.

17. A method for gathering information on a circulating blood of an animal, including humans, wherein said method comprises the following steps:
1) providing a holding and containing device in fixed position with respect to said animal, including humans, adapted to hold, contain and maintain in fixed position a minimum of one element, where said minimum of one element is at least one or more from a set of elements composed of:
1a) a minimum of one radiation emitting device emitting a radiation along a first propagation path,
1b) a minimum of one radiation detection device detecting radiation along a second propagation path and within a cone of acceptance,
1c) electronics circuits,
2) where each of said minimum of one radiation detection device is positioned so as to detect said radiation emitted by said radiation emitting device that have suffered one or more than one scattering events inside said animal and after said one or more scattering event, propagates along such propagation path that makes an angle larger than zero degrees and less then 180 degrees with said first propagation path of said radiation emitted by said radiation emitting device,
3) fixing said minimum of one radiation emitting device at such locations with respect to said holding and containing device, that said radiation emitted by said radiation emitting device propagates inside said animal, including humans, along a direction that makes an angle larger than 1 degree and smaller than 75 degrees with a skin of said animal located at the closest proximity to said propagating radiation and at a depth of less than 1 cm from said first surface of said skin of said animal located at said closest proximity to said propagating radiation of said animal,
4) where said holding and containing device is formed with a re-entrant cavity where said radiation emitted by said radiation emitting device propagates to be detected and measured by said radiation detecting device, such that, when said holding and containing device is fixed in position, a flesh of said animal, including human , is pressed to fill in a space of said re-entrant cavity.

18. The method of claim 17, where said re-entrant cavity is created on said holding and containing device by a cavity on said holding and containing device on a side of said holding and containing device that is facing said flesh of said animal.

19. The method of claim 17, where said re-entrant cavity is created on said holding and containing device by a pair of protruding elements on said holding and containing device, on a side of said holding and containing device that is facing said flesh of said animal.

20. The method of claim 17, where said re-entrant cavity is created on said holding and containing device by a single protruding element on said holding and containing device, on a side of said holding and containing device that is facing said flesh of said animal.
